(19) United States
(12) Patent Application Publication
(10) Pub. No.: US 2023/0212193 A1
(43) Pub. Date: Jul. 6, 2023

(54) CRYSTALLINE RET INHIBITOR

(71) Applicant: Loxo Oncology, Inc., Indianapolis, IN (US)

(72) Inventors: Rajni Miglani BHARDWAJ, Zionsville, IN (US); Jeremy Miles MERRITT, Indianapolis, IN (US); Jon Gordon SELBO, Noblesville, IN (US); Shekhar Krishna VISWANATH, Carmel, IN (US)

(21) Appl. No.: 17/996,028

(22) PCT Filed: Apr. 9, 2021
(86) PCT No.: PCT/US2021/026611

§ 371 Date: Oct. 12, 2022

Related U.S. Application Data
(60) Provisional application No. 63/011,701, filed on Apr. 17, 2020.

(51) Int. Cl. C07D 519/00

(52) U.S. Cl. CPC C07D 519/00; C07B 2200/13

(57) ABSTRACT

Provided herein is a crystalline form of selpercatinib useful in the treatment and prevention of diseases which can be treated with a RET kinase inhibitor, including RET-associated diseases and disorders, and methods of making this crystalline form.

### BACKGROUND

Selpercatinib (LOXO-292 or RETEVMO™) is a RET inhibitor approved in the United States for use in the treatment of patients with metastatic RET fusion-positive NSCLC, RET-mutant medullary thyroid cancer, and RET fusion-positive thyroid cancer. Selpercatinib, or 6-(2-hydroxy-2-methylpropoxy)-4-(6-(6-((6-methoxypyridin-3-yl)methyl)-3,6-diazabicyclo[3.1.1]heptan-3-yl)pyridin-3-yl)pyrazolo[1,5-a]pyridine-3-carbonitrile, has the following chemical structure:

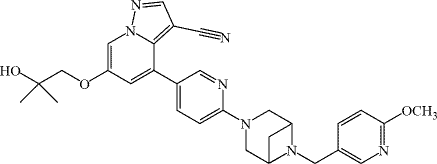 [SMILES: COc1ccc(CN2C3CC2CN(c2ccc(-c4cc(OCC(C)(C)O)cn5ncc(C#N)c45)cn2)C3)cn1]

While U.S. Pat. No. 10,584,124 describes several crystal forms for selpercatinib, including a crystalline form referred to as “Form A,” disclosed herein is a new, thermodynamically more stable crystal form, and methods of making this crystal form. This new crystal form can be incorporated into formulations, such as tablets, capsules, and suspensions, which would benefit patients.

### SUMMARY

This disclosure relates to a new crystalline form of selpercatinib, and methods of making this thermodynamically stable polymorph, which is referred to throughout as “Form B.” In a general sense, this disclosure provides methods for its preparation, isolation, and characterization.

As described in more detail below, the compound of Formula I (selpercatinib) can be provided as polymorphic forms (Form A and Form B) and, surprisingly, that certain processes and methods are effective to provide selpercatinib in its most thermodynamically stable polymorph Form B. As described below and demonstrated by the illustrative working examples, the processes and methods for generating and preparing selpercatinib in a specific polymorph form may comprise converting (i.e., reacting, contacting, and/or treating) the compound of Formula I provided as one or more polymorph forms, under crystallization conditions that are effective to generate or convert the other polymorphs (i.e., Form A) to Form B. In other aspects, the processes and methods for generating selpercatinib Form B may comprise a synthetic route comprising reacting one or more intermediate or precursor compounds under conditions that are effective to generate selpercatinib Form B (i.e., direct synthetic routes).

Form B is characterized by at least one of (a) an x-ray powder diffraction (XRPD) pattern comprising a peak at 21.1 ° and one or more peaks at 17.1°, 17.7°, and 19.8° ± 0.2° 2θ as measured using an x-ray wavelength of 1.5418 Å, or (b) a ¹³C solid state NMR spectrum which comprises peaks referenced to the high field resonance of adamantane (δ = 29.5 ppm) at: 28.0, 48.0, 80.4, 106.8, 130.2, and 134.9 ppm (± 0.2 ppm, respectively).

Methods of using Form B and pharmaceutical compositions thereof, to treat cancer, such as cancers with abnormal RET expression (e.g., a RET-associated cancer like medullary thyroid cancer or RET fusion lung cancer) are also provided. The methods include administering a therapeutically effective amount of Form B to a patient in need.

Also provided herein, is Form B for use in therapy. Further provided herein, is Form B for use in the treatment of cancer, in particular, for use in the treatment of cancer with abnormal RET expression (e.g., a RET-associated cancer like medullary thyroid cancer or RET fusion lung cancer).

The use of Form B in the manufacture of a medicament for treating cancer, in particular, for use in the treatment of cancer with abnormal RET expression (e.g., a RET-associated cancer like medullary thyroid cancer or RET fusion lung cancer), is also provided.

Methods of converting selpercatinib Form A to selpercatinib Form B are also disclosed.

A method for converting selpercatinib Form A to selpercatinib Form B, the method comprising: combining selpercatinib Form A with a C₁-C₅ alcohol to generate a slurry and isolating selpercatinib Form B from the slurry, is also detailed herein.

Also described is a method for converting selpercatinib Form A to selpercatinib Form B, the method comprising:
a. dissolving the selpercatinib Form A in a solvent comprising DMSO to form a solution;
b. adding water to the solution and thereby forming a slurry;
c. isolating the selpercatinib Form B.

Further described is a method for converting selpercatinib Form A to form b, the method comprising: combining selpercatinib Form A and methanol to form a slurry, and stirring the slurry until >99 wt% of the Form A is converted to Form B.

Another method described herein is a method for converting selpercatinib Form A to Form B, wherein the selpercatinib Form A is dissolved in DMSO at about 60-80° C. to form a solution having a concentration of about 10-15 mL/g of DMSO per gram of Form A; cooling the solution to about 40-60° C., adding water; optionally seeding the resulting mixture with Form B seed crystals; stirring the mixture; adding more water; heating the mixture to about 60-80° C.; cooling the mixture and isolating the Form B.

A process for preparing selpercatinib as polymorph Form B, of Formula I:

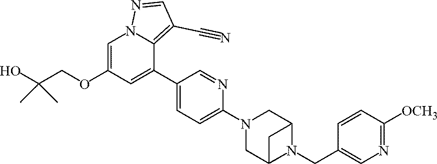 [SMILES: COc1ccc(CN2C3CC2CN(c2ccc(-c4cc(OCC(C)(C)O)cn5ncc(C#N)c45)cn2)C3)cn1]

or a pharmaceutically acceptable salt thereof, wherein the process comprises reacting a compound of the structure:

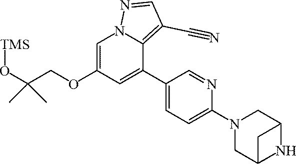 [SMILES: CC(C)(COc1cc(-c2ccc(N3CC4CC(C3)N4)nc2)c2c(C#N)cnn2c1)O[Si](C)(C)C]

or a salt thereof, in a solvent with 6-methoxynicotinaldehyde in the presence of an acid and a reducing agent to prepare selpercatinib Form B or a pharmaceutically acceptable salt thereof, is also described.

A compound that is 4-[6-(3,6-diazabicyclo[3.1.1]heptan-3-yl)-3-pyridyl]-6-(2-methyl-2-trimethylsilyloxy-propoxy)pyrazolo[1,5-a]pyridine-3-carbonitrile, which has the structure [3]

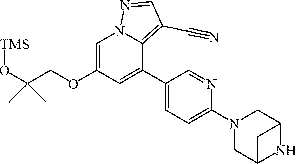 [SMILES: CC(C)(COc1cc(-c2ccc(N3CC4CC(C3)N4)nc2)c2c(C#N)cnn2c1)O[Si](C)(C)C]

or a pharmaceutically acceptable salt thereof, is described herein.

### BRIEF DESCRIPTION OF THE FIGURES

FIG. 1 is an overlay of Form A and Form B XRPD data, up to about 26 ° two theta (2 Θ).

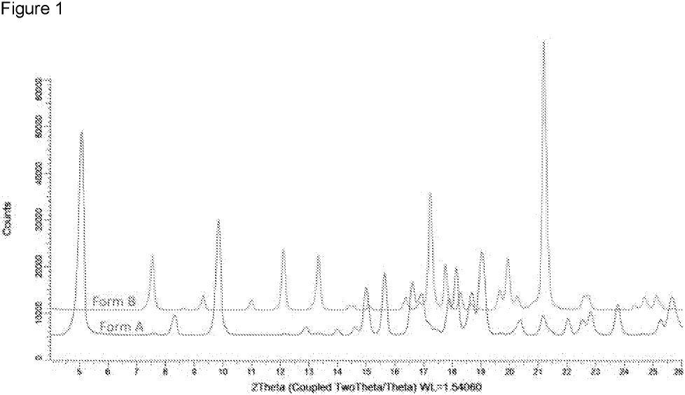

FIG. 2 contains ¹³C solid state NMR data for Form A, Form B, and an overlay of about 25 to 60 ppm that compares Form A to Form B.

### DETAILED DESCRIPTION

Selpercatinib Form B is described herein. This crystalline form of selpercatinib could be used to treat disorders associated with abnormal RET activity, e.g., IBS or cancer, especially cancer stemming from overactive RET signaling (i.e., RET-associated cancers). More specifically, this crystalline form of selpercatinib could be used to treat RET-associated cancers such as lung cancer (e.g., small cell lung carcinoma or non-small cell lung carcinoma), thyroid cancer (e.g., papillary thyroid cancer, medullary thyroid cancer, differentiated thyroid cancer, recurrent thyroid cancer, or refractory differentiated thyroid cancer), thyroid ademona, endocrine gland neoplasms, lung adenocarcinoma, bronchioles lung cell carcinoma, multiple endocrine neoplasia type 2A or 2B (MEN2A or MEN2B, respectively), pheochromocytoma, parathyroid hyperplasia, breast cancer, mammary cancer, mammary carcinoma, mammary neoplasm, colorectal cancer (e.g., metastatic colorectal cancer), papillary renal cell carcinoma, ganglioneuromatosis of the gastroenteric mucosa, inflammatory myofibroblastic tumor, or cervical cancer.

Form B is characterized by having an x-ray powder diffraction (XRPD) pattern comprising a peak at 21.1 ° and one or more peaks at 17.1 °, 17.7°, and 19.8° ± 0.2° 2θ as measured using an x-ray wavelength of 1.5418 Å. Form B also exhibits a ¹³C solid state NMR spectrum comprising peaks referenced to the high field resonance of adamantane (δ = 29.5 ppm) at 28.0, 48.0, 80.4, 106.8, 130.2, and 134.9 ppm (± 0.2 ppm, respectively).

Form B can be further characterized by having an x-ray powder diffraction (XRPD) pattern comprising a peak at 21.1 ° and one or more peaks occurring at 7.5°, 12.0°, 13.2°, 17.1°, 17.7°, and 19.8° ± 0.2° 2θ as measured using an x-ray wavelength of 1.5418 Å.

Additionally, Form B can be characterized by having an x-ray powder diffraction (XRPD) pattern comprising a peak at 21.1 ° and one or more peaks occurring at 7.5°, 10.9°, 12.0°, 13.2°, 17.1°, 17.7°, 18.2°, 19.8°, 21.1°, and 24.5° ± 0.2° 2θ as measured using an x-ray wavelength of 1.5418 Å.

Form B can be further characterized by a ¹³C solid state NMR spectrum which comprises peaks referenced to the high field resonance of adamantane (δ = 29.5 ppm) at: 26.4, 28.0, 42.0, 43.9, 48.0, 56.3, 69.5, 80.4, 102.3, 106.8, 115.2, 120.8, 130.2, 134.9, 140.6, 149.5, 152.5, and 163.5 ppm (± 0.2 ppm, respectively).

Moreover, Form B can be characterized by a 13C solid state NMR spectrum which comprises one or more peaks referenced to the high field resonance of adamantane (δ = 29.5 ppm) at: 26.4, 27.4, 28.0, 42.0, 43.4, 43.9, 48.0, 53.9, 56.3, 58.3, 69.5, 77.9, 80.4, 102.3, 106.8, 113.6, 115.2, 118.2, 120.8, 125.2, 130.2, 134.9, 136.9, 140.6, 148.4, 149.5, 151.2, 152.5, 158.2, 163.5 ppm (+ 0.2 ppm, respectively).

Also described herein are pharmaceutical composition comprising Form B, and one or more pharmaceutically acceptable carriers, diluents, or excipients.

A pharmaceutical composition containing Form B includes at least 5%, 10%, 20%, 30%, 40%, 50%, 60%, 70%, 80%, 90%, 95%, 96%, 97%, 98%, 99% or 100% by weight of Form B, as compared to other crystal forms of selpercatinib. Preferably the pharmaceutical compositions described herein comprise at least 80% of Form B and less than 20% of other crystal forms of selpercatinib. More preferably the pharmaceutical compositions comprise at least 90% of Form B and less than 10% of other crystal forms of selpercatinib. Even more preferably the pharmaceutical compositions comprise at least 95% Form B and less than 5% of other crystal forms of selpercatinib. Still more preferably, the pharmaceutical compositions comprise at least 97% Form B and less than 3% of other crystal forms of selpercatinib. More preferably, the pharmaceutical compositions comprise at least 98% or 99% Form B and less than 2% or 1%, respectively, of other crystal forms of selpercatinib.

Form B may be used in a method for treating cancer, comprising administering an effective amount of Form B to a patient in need thereof. The types of cancers that may be treated using the methods described herein include hematological cancer or solid tumor cancer. Examples of the types of cancer that may be treated using Form B include lung cancer, papillary thyroid cancer, medullary thyroid cancer, differentiated thyroid cancer, recurrent thyroid cancer, refractory differentiated thyroid cancer, multiple endocrine neoplasia type 2A or 2B (MEN2A or MEN2B, respectively), pheochromocytoma, parathyroid hyperplasia, breast cancer, colorectal cancer, papillary renal cell carcinoma, ganglioneuromatosis of the gastroenteric mucosa, and cervical cancer. Specifically, the types of cancer can be lung cancer or thyroid cancer. More specifically, the cancer can be non-small cell lung carcinoma or medullary thyroid cancer.

Also described herein is Form B, for use in therapy.

Form B may be used in the manufacture of a medicament for the treatment of RET-associated diseases or disorders such as IBS or cancer. Cancers that can be treated using such a medicament are described herein above. Use of Form B in the manufacture of a medicament may also include a step of performing an in vitro assay using a biological sample from a patient, determining the presence of a dysregulation of a RET gene, a RET kinase, or expression or activity or level of any of the same, and administering a therapeutically effective amount of Form B, to the patient if a dysregulation of a RET gene, a RET kinase, or expression or activity or level of any of the same is present. In these uses, the biological sample can be a tumor sample and the tumor sample can be analyzed using methods known to those of skill in the art such as genomic/DNA sequencing. Additionally, in these uses the sample can be obtained from the patient prior to the first administration of Form B. In these uses of Form B, as described herein in a therapy can be based upon a patient being selected for treatment by having at least one dysregulation of a RET gene, a RET kinase, or expression or activity or level of any of the same. Also, in these uses Form B may be administered to the patient at a dose of about 1 mg/kg to 200 mg/kg (effective dosage sub-ranges are noted herein above).

Herein, a patient is one in whom a RET fusion or RET mutation has been determined. As such, the term “determining a RET fusion or RET mutation” means determining if a RET fusion or RET mutation is present. Methods for determining the if a RET fusion or RET mutation is present are known to those of ordinary skill in the art, e.g., see Wang, Yucong et al., Medicine 2019; 98(3): e14120.

As used above, and throughout the description of the invention, the following terms, unless otherwise indicated, shall be understood to have the following meanings:

A “pharmaceutically acceptable carrier, diluent, or excipient” is a medium generally accepted in the art for the delivery of biologically active agents to mammals, e.g., humans.

The terms “treatment,” “treat,” “treating,” and the like, are meant to include slowing, stopping, or reversing the progression of a disorder. These terms also include alleviating, ameliorating, attenuating, eliminating, or reducing one or more symptoms of a disorder or condition, even if the disorder or condition is not actually eliminated and even if progression of the disorder or condition is not itself slowed, stopped, or reversed.

“Effective amount” means the amount of the crystalline form of selpercatinib that will elicit the biological or medical response of or desired therapeutic effect on a patient by a treating clinician. In one example, the crystalline form of selpercatinib inhibits native RET signaling in an in vitro or ex vivo RET enzyme assay. In another example, the crystalline form of selpercatinib inhibits native RET signaling in mouse whole blood from animals treated with different doses of the compound.

As used herein, the term “patient” refers to a human.

An effective amount can be readily determined by the attending diagnostician, as one skilled in the art, by the use of known techniques and by observing results obtained under analogous circumstances. In determining the effective amount for a patient, a number of factors are considered by the attending diagnostician, including, but not limited to: the species of patient; its size, age, and general health; the specific disease or disorder involved; the degree of or involvement or the severity of the disease or disorder; the response of the individual patient; the particular compound administered; the mode of administration; the bioavailability characteristics of the preparation administered; the dose regimen selected; the use of concomitant medication; and other relevant circumstances.

Form B is preferably formulated as pharmaceutical compositions administered by any route which makes the compound bioavailable, including oral, intravenous, and transdermal routes. Most preferably, such compositions are for oral administration. Such pharmaceutical compositions and processes for preparing same are well known in the art. (See, e.g., Remington: The Science and Practice of Pharmacy (D.B. Troy, Editor, 21st Edition, Lippincott, Williams & Wilkins, 2006).

As used herein, “granulate composition” refers to a composition in granular form which, in the pharmaceutical manufacturing process, is a predecessor composition to a pharmaceutical composition.

As used herein, “manufacturing container” refers to a container that is employed in the manufacture of a pharmaceutical, but not in the medicinal chemistry laboratory. Examples of manufacturing containers include, but are not limited to, a hopper collector, a bed, a dryer bed, a granulator bed, a dryer tray, a granulator bucket, and a mixing bowl.

In some embodiments, the Form B material is prepared from the Form A material. In an embodiment, the method of converting Form A to Form B comprises: combining selpercatinib Form A with a C₁-C₅ alcohol to generate a slurry and isolating selpercatinib Form B from the slurry. In some embodiments, the method is performed at a temperature of about 10-80° C., about 10-30° C., about 15-25° C., or about 20° C.

In some embodiments the C₁-C₅ alcohol comprises methanol. Preferred C₁-C₅ alcohols comprise methanol, and in some embodiments, the methanol is at least about 90 wt%, or 92 wt%, or 94 wt%, or 96 wt %, or 98 wt%, or 99 wt% methanol.

In other embodiments, the method comprises: combining selpercatinib Form A with water to generate a slurry and isolating selpercatinib Form B from the slurry. In some embodiments, the method is performed at a temperature of about 10-80° C., about 10-30° C., about 15-25° C., or about 20° C.

In some embodiments, the method comprises stirring, mixing, or agitating the slurry for a period of time ranging from at least about 5 minutes (e.g., at least 5, 6, 7, 8, 9, 10, 11, 12, 13, 14, 15, 16, 17, 18, 19, 20, 25, 30, 35, 40, 45, 50, 55, or at least 60 minutes). In some embodiment the period of time may be about 8-12 hours. In some further embodiments, the period of time is at least 10 minutes.

In some embodiments, the method may further comprise isolating selpercatinib Form B that is generated by the method. In some embodiments the isolating may comprise vacuum filtration. In some embodiments the isolating may comprise centrifugal separation.

In some further embodiments, the method may further comprise drying the selpercatinib Form B that is generated. Drying may be accomplished using vacuum and/or thermal means.

In other embodiments, the method comprises dissolving selpercatinib Form A in a solvent comprising DMSO to form a solution; adding water to the solution in an amount to form a slurry; and isolating the selpercatinib Form B generated in the slurry.

In some embodiments, the method comprises adding about 1 gram of selpercatinib Form A to about 10-15 mL/g of DMSO. In some further embodiments, the method comprises adding about 1 equivalent of selpercatinib form in about 12-13 mL/g of DMSO, and thus, the concentration of Form A dissolved in DMSO is about 12-13 mL/g or 1 g of Form A in about 12-13 mL of DMSO.

In some embodiments of the method, forming the solution comprising DMSO and selpercatinib Form A comprises heating the selpercatinib Form A and the solvent comprising DMSO to about 50° C. to about 70° C. In some further embodiments, the method comprises cooling the solution to a temperature less than about 70° C. and greater than about 20° C. In yet further embodiments, the method comprises cooling the solution to a temperature of about 50° C.

In some embodiments of the method, the adding of water comprises adding about 0.1 to about 1 mL/g of water to the solution per gram of Form A. In some further embodiments, the addition of water comprises adding about 0.3 mL/g of water per gram of Form A to the solution.

In some embodiments of the method, the adding of water may further comprise adding about 1 to about 15 wt% of Form B seed crystals to the slurry. In some further embodiments, about 1 to about 10 wt% of Form B seed crystals may be added to the slurry. In yet further embodiments, about 5 wt% of Form B seed crystals may be added to the slurry.

In some embodiments of the method, the slurry is stirred for about 6 to about 72 hours, after the water is added. In some embodiments, the slurry is stirred for at least 12 hours.

In some embodiments the method may further comprise a second addition of water to the slurry formed by the first addition of water. In some embodiments, the second addition of water may be added to the slurry in an amount of about 0.5 to about 3 mL/g of water are added to the slurry.

In some embodiments of the method, wherein the slurry formed by the adding of water is cooled to about 20-30° C.

In some embodiments, the isolating of the selpercatinib Form B comprises filtration. In some embodiments, the isolated selpercatinib Form B may be washed with a solvent comprising methanol, ACN, MTBE, or water. In some further embodiments, the isolated selpercatinib Form B is washed with a solvent comprising methanol. In yet further embodiments, the isolated selpercatinib Form B is washed with methanol until the isolated selpercatinib Form B contains less than 0.5 wt % DMSO.

In some of the above aspects and embodiments, the disclosure provides a method for converting selpercatinib Form A to Form B, comprising: combining selpercatinib Form A and methanol to form a slurry, and stirring the slurry until >99 wt% of the Form A is converted to Form B. In some embodiments of the method, the slurry is stirred for about 18-24 hours. In yet further embodiments, the concentration of the selpercatinib Form A in the methanol is about 8 mL/g.

In some of the above aspects and embodiments, the disclosure provides a method for converting selpercatinib Form A to Form B, wherein the selpercatinib Form A is dissolved in DMSO at about 60-80° C. to form a solution having a concentration of about 10-15 mL/g of DMSO per gram of Form A; cooling the solution to about 40-60° C., adding a first amount of water; optionally seeding the resulting mixture with Form B seed crystals; stirring the mixture; adding a second amount of water; heating the mixture to about 60-80° C.; cooling the mixture and isolating the Form B. In some embodiments of the method 5 wt% of Form B seed crystals are added to the mixture. In yet further embodiments, the first addition of water is about 0.1 mL/g of Form A to about 0.5 mL/g of Form A. In still further embodiments, the second addition of water is about 1.0-1.5 mL/g of Form A.

In another aspect, the disclosure provides a process for preparing selpercatinib as polymorph Form B, of Formula I:

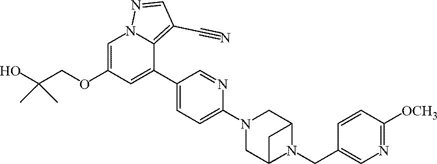 [SMILES: COc1ccc(CN2C3CC2CN(c2ccc(-c4cc(OCC(C)(C)O)cn5ncc(C#N)c45)cn2)C3)cn1]

or a pharmaceutically acceptable salt thereof, wherein the process comprises reacting a compound of the structure:

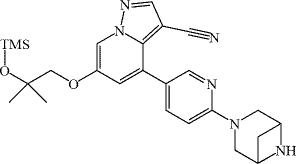 [SMILES: CC(C)(COc1cc(-c2ccc(N3CC4CC(C3)N4)nc2)c2c(C#N)cnn2c1)O[Si](C)(C)C]

or a salt thereof, in a solvent with 6-methoxynicotinaldehyde in the presence of an acid and a reducing agent to prepare selpercatinib Form B or a pharmaceutically acceptable salt thereof. While a stoichiometric amount of acid may be used, non-stoichiometric amounts are also acceptable. In structure [3], the oxygen has a TMS group on it. While not expressly shown, it is understood that other alcohol protecting groups could be used. Besides TMS, other silyl groups can be used, as described herein.

In some embodiments of this aspect, the process further comprises preparing a compound of structure [3], or a salt thereof, comprising reacting a compound of the structure

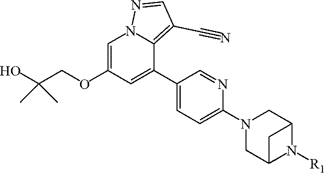

or a salt thereof, wherein R₁ is an amine protecting group, with a deprotecting agent to form the compound of structure [3] or a salt thereof.

In some embodiments, the deprotecting agent is selected from the group consisting of trifluoroacetic acid, hydrochloric acid, hydrobromic acid, hydriodic acid, phosphoric acid, sulfuric acid, methanesulfonic acid, p-toluene sulfonic acid, acetyl chloride, aluminum trichloride, and boron trifluoride. In some further embodiments, the deprotecting agent is selected from the group consisting of sulfuric acid, p-toluene sulfonic acid, and acetyl chloride.

In some embodiments, the reducing agent is selected from the group consisting of an alkali metal borohydride, a hydrazine compound, citric acid, a citric acid salt, succinic acid, a succinic acid salt, ascorbic acid, and an ascorbic acid salt. In some further embodiments, the reducing agent is selected from the group consisting of sodium triacetoxyborohydride (STAB), sodium borohydride, and sodium cyanoborohydride.

In some embodiments, R₁ is selected from the group consisting of formyl, acetyl, trifluoroacetyl, benzyl, benzoyl, carbamate, benzyloxycarbonyl, p-methoxybenzyl carbonyl, tert-butyloxycarbonyl (Boc), trimethylsilyl, 2-trimethylsilyl-ethanesulfonyl, trityl and substituted trityl groups, allyloxycarbonyl, 9-fluorenylmethyloxycarbonyl, nitroveratryloxycarbonyl, p-methoxybenzyl, and tosyl. In some further embodiments, R₁ is tert-butyloxycarbonyl (Boc).

In some embodiments, the acid is selected from the group consisting of pivalic acid and acetic acid. In some further embodiments, the acid is pivalic acid. In a still further embodiment, a catalytic amount of pivalic acid is used.

In some embodiments, the reacting of compound [3] is performed in an aprotic solvent. Examples of a protic solvents include ethers, such as anisole.

In another aspect, the disclosure provides a compound 4-[6-(3,6-diazabicyclo[3.1.1]heptan-3-yl)-3-pyridyl]-6-(2-methyl-2-trimethylsilyloxy-propoxy)pyrazolo[1,5-a]pyridine-3-carbonitrile of the structure [3]:

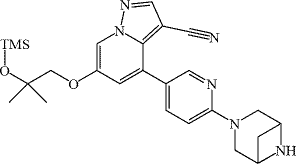 [SMILES: CC(C)(COc1cc(-c2ccc(N3CC4CC(C3)N4)nc2)c2c(C#N)cnn2c1)O[Si](C)(C)C]

or a pharmaceutically acceptable salt thereof.

In some embodiments, the disclosure provides a method for preparing the compound of structure [3], in accordance with the aspects and embodiments described herein.

In some embodiments of any of the above aspects, the method comprises preparing selpercatinib Form B as the free amine.

Selpercatinib Form A (Form A) can contain some of its thermodynamically more stable polymorph selpercatinib Form B (Form B). While both polymorph forms are crystalline, high-melting, anhydrous, stable, and do not inter-convert under typical storage or preparative conditions, the polymorphs have different properties and characteristics, which allows Form A to be distinguished from Form B. Since Form B is thermodynamically more stable, there is a need to understand how to convert Form A to Form B.

### Definitions

Unless defined otherwise, all technical and scientific terms used herein have the meaning commonly understood by a person skilled in the art to which this invention belongs. As used herein, the following terms have the meanings ascribed to them below, unless specified otherwise.

The term “polymorph,” as used herein, refers to crystals of the same compound having different physical properties as a result of the order of the molecules in the crystal lattice. Different polymorphs of a single compound (i.e. a compound of Formula I) have one or more different chemical, physical, mechanical, electrical, thermodynamic, and/or biological properties from each other. Differences in physical properties exhibited by polymorphs can affect pharmaceutical parameters such as storage stability, compressibility, density (important in composition and product manufacturing), dissolution rates (an important factor in determining bioavailability), solubility, melting point, chemical stability, physical stability, powder flowability, water sorption, compaction, and particle morphology. Differences in stability can result from changes in chemical reactivity (e.g., differential oxidation, such that a dosage form discolors more rapidly when comprised of one polymorph than when comprised of another polymorph) or mechanical changes (e.g., crystal changes on storage as a kinetically favored polymorph converts to a thermodynamically more stable polymorph) or both (e.g., one polymorph is more hygroscopic than the other). As a result of solubility/dissolution differences, some transitions affect potency and/or toxicity. In addition, the physical properties of the crystal may be important in processing; for example, one polymorph might be more likely to form solvates or might be difficult to filter and wash free of impurities (i.e., particle shape and size distribution might be different between one polymorph relative to the other). “Polymorph”, as used herein, does not include amorphous forms of the compound. In some particular embodiments, the polymorph of the compound of Formula I (i.e., selpercatinib Form A and selpercatinib Form B) comprises the characteristics as described herein.

As used herein, “amorphous” refers to a noncrystalline form of a compound which can be a solid state form of the compound or a solubilized form of the compound. For example, “amorphous” refers to a compound (e.g., a solid form of the compound) without a regularly repeating arrangement of molecules or external face planes.

The term “anhydrous,” as used herein, refers to a crystal form of the compound of Formula (I) that does not contain stoichiometric amounts of water associated with the crystal lattice. Typically, anhydrous Form A and anhydrous Form B have 1% or less by weight water. For example, 0.5% or less, 0.25% or less, or 0.1% or less by weight water.

The term “solvate” as used herein refers to a crystalline form of the compound of Formula (I), where the crystal lattice includes one or more solvents.

The terms “hydrate” or “hydrated polymorph form” refer to a crystalline form of the compound of Formula (I), such as a polymorph form of the compound, where the crystal lattice includes water. Unless specified otherwise, the term “hydrate” as used herein refers to a “stoichiometric hydrate.” A stoichiometric hydrate contains the water molecules as an integral part of the crystal lattice. In comparison, a non-stoichiometric hydrate comprises water, but changes in the water content does not cause significant changes to the crystal structure. During drying of non-stoichiometric hydrates, a considerable proportion of water can be removed without significantly disturbing the crystal network, and the crystals can subsequently rehydrate to give the initial non-stoichiometric hydrated crystalline form. Unlike stoichiometric hydrates, the dehydration and rehydration of non-stoichiometric hydrates is not accompanied by a phase transition, and thus all hydration states of a non-stoichiometric hydrate represent the same crystal form.

“Purity,” when used in reference to a composition including a polymorph of the compound of Formula (I), refers to the percentage of one specific polymorph form relative to another polymorph form or an amorphous form of the compound of Formula (I) in the referenced composition. For example, a composition comprising polymorph Form 1 having a purity of 90% would comprise 90 weight parts Form 1 and 10 weight parts of other polymorph and/or amorphous forms of the compound of Formula (I).

As used herein, a compound or composition is “substantially free of” one or more other components if the compound or composition contains no significant amount of such other components. For example, the composition can contain less than 5%, 4%, 3%, 2%, or 1% by weight of other components. Such components can include starting materials, residual solvents, or any other impurities that can result from the preparation of and/or isolation of the compounds and compositions provided herein. In some embodiments, a polymorph form provided herein is substantially free of other polymorph forms. In some embodiments, a particular polymorph of the compound of Formula (I) is “substantially free” of other polymorphs if the particular polymorph constitutes at least about 95% by weight of the compound of Formula (I) present. In some embodiments, a particular polymorph of the compound of Formula (I) is “substantially free” of other polymorphs if the particular polymorph constitutes at least about 97%, about 98%, about 99%, or about 99.5% by weight of the compound of Formula (I) present. In certain embodiments, a particular polymorph of the compound of Formula (I) is “substantially free” of water if the amount of water constitutes no more than about 2%, about 1%, or about 0.5% by weight of the polymorph.

As used herein, “substantially pure,” when used in reference to a polymorph form of the compound of Formula (I), means a sample of a polymorph form of the compound having a purity greater than 90%, including greater than 90%, 91%, 92%, 93%, 94%, 95%, 96%, 97%, 98%, and 99%, and also including equal to about 100% of the compound, based on the weight of the compound. The remaining material comprises other form(s) of the compound, and/or reaction impurities and/or processing impurities arising from its preparation. For example, a polymorph form of the compound of Formula (I) may be deemed substantially pure in that it has a purity greater than 90% of a polymorph form of the compound of Formula (I), as measured by means that are at this time known and generally accepted in the art, where the remaining less than 10% of material comprises other form(s) of the compound of Formula (I) and/or reaction impurities and/or processing impurities. The presence of reaction impurities and/or processing impurities may be determined by analytical techniques known in the art, such as, for example, chromatography, nuclear magnetic resonance spectroscopy, mass spectrometry, or infrared spectroscopy.

To provide a more concise description, some of the quantitative expressions herein are recited as a range from about amount X to about amount Y. It is understood that when a range is recited, the range is not limited to the recited upper and lower bounds, but rather includes the full range from about amount X through about amount Y, or any range therein.

“Room temperature” or “RT” refers to the ambient temperature of a typical laboratory, which is typically around 25° C.

As used herein, the term “excipient” refers to any substance needed to formulate the composition to a desired form. For example, suitable excipients include but are not limited to, diluents or fillers, binders or granulating agents or adhesives, disintegrants, lubricants, antiadherants, glidants, dispersing or wetting agents, dissolution retardants or enhancers, adsorbents, buffers, chelating agents, preservatives, colors, flavors and sweeteners.

The term “pharmaceutically acceptable carrier” or “pharmaceutically acceptable excipient” includes any and all solvents, co-solvents, complexing agents, dispersion media, coatings, antibacterial and antifungal agents, isotonic and absorption delaying agents, and the like, which are not biologically or otherwise undesirable. The use of such media and agents for pharmaceutically active substances is well known in the art. Except insofar as any conventional media or agent is incompatible with the active ingredient, its use in the therapeutic formulations is contemplated. Supplementary active ingredients can also be incorporated into the formulations. In addition, various excipients, such as are commonly used in the art, can be included. These and other such compounds are described in the literature, e.g., in the Merck Index, Merck & Company, Rahway, N.J. Considerations for the inclusion of various components in pharmaceutical compositions are described, e.g., in Gilman et al. (Eds.) (2010); Goodman and Gilman’s: The Pharmacological Basis of Therapeutics, 12th Ed., The McGraw-Hill Companies.

As used herein, the singular forms “a,” “an,” and “the” include plural referents unless the context clearly dictates otherwise.

As used herein, ranges and amounts can be expressed as “about” a particular value or range. About also includes the exact amount. Hence, “about 5 grams” means “about 5 grams” and also “5 grams.” It also is understood that ranges expressed herein include whole numbers within the ranges and fractions thereof. For example, a range of between 5 grams and 20 grams includes whole number values such as 5, 6, 7, 8, 9, 10, 11, 12, 13, 14, 15, 16, 17, 18, 19 and 20 grams, and fractions within the range including, but not limited to, 5.25, 6.5, 8.75 and 11.95 grams. The term “about” preceding a value for DSC, TGA, TG, or DTA, which are reported as degrees Celsius, have an allowable variability of +/-5° C.

As used herein, “optional” or “optionally” means that the subsequently described event or circumstance does or does not occur and that the description includes instances where said event or circumstance occurs and instances where it does not. For example, a reaction mixture that “optionally includes a catalyst” means that the reaction mixture contains a catalyst or it does not contain a catalyst.

As used herein, “strong base” refers to a basic chemical compound that is able to deprotonate weak acids in an acid-base reaction. Examples of strong bases include, but are not limited to, hydroxides, alkoxides, and ammonia. Common examples of strong bases are the hydroxides of alkali metals and alkaline earth metals, e.g., NaOH. Certain strong bases are even able to deprotonate very weakly acidic C--H groups in the absence of water. Strong bases include, but are not limited to, sodium hydroxide, potassium hydroxide, barium hydroxide, cesium hydroxide, calcium hydroxide, strontium hydroxide, lithium hydroxide and rubidium hydroxide. In some embodiments, NaOH is used as the strong base. In some embodiments, potassium hydroxide is used as the strong base.

As used herein, the term “weak base” refers to inorganic and organic bases that are only partially ionized in aqueous solution. Weak bases typically have a pKa of between about 6 and about 11. A large number of such weak bases are known and are exemplified by those listed in the Handbook of Biochemistry and Molecular Biology, Vol. 1, 3rd ed., G. D. Fassman, CRC Press, 1976, pp. 305-347. The weak base can be soluble or insoluble in water. Suitable weak bases include, but are not limited to, alkali metal carbonates and bicarbonates, such as lithium carbonate, sodium carbonate, potassium carbonate, cesium carbonate, and sodium bicarbonate; ammonia; primary amines, such as methylamine; secondary amines; and tertiary amines, such as the trialkylamines, e.g., trimethylamine, triethylamine, tripropylamine and tributylamine, benzyldiethylamine, pyridine, quinoline, N-methylmorpholine, aniline, and the like.

“Non-nucleophilic base,” as used herein, refers to a base that will not act as a nucleophile, i.e., a base that will not donate an electron pair to an electrophile to form a chemical bond in relation to a reaction. Typically, non-nucleophilic bases are bulky and sterically hindered, such that protons can attach to the basic center, but alkylation and complexation are prevented. Examples of non-nucleophilic bases include, but are not limited to, amines and nitrogen heterocycles, such as triethylamine and pyridine, amidines, lithium compounds, and phosphazenes. Other examples of non-nucleophilic bases include sodium hydride and potassium hydride.

As used herein, the term “amine protecting group” means any group known in the art of organic synthesis for the protection of amine groups. Such amine protecting groups include those listed in Greene, “Protective Groups in Organic Synthesis,” John Wiley & Sons, New York (1981) and “The Peptides: Analysis, Synthesis, Biology, Vol. 3,” Academic Press, New York (1981). Any amine protecting group known in the art can be used. Examples of amine protecting groups include, but are not limited to, the following: (1) acyl types such as formyl, trifluoroacetyl, phthalyl, and p-toluenesulfonyl; (2) aromatic carbamate types such as benzyloxycarbonyl (Cbz) and substituted benzyloxycarbonyls, 1-(p-biphenyl)-1-methylethoxycarbonyl, and 9-fluorenylmethyloxycarbonyl (Fmoc); (3) aliphatic carbamate types such as tert-butyloxycarbonyl (Boc), ethoxycarbonyl, diisopropylmethoxycarbonyl, and allyloxycarbonyl; (4) cyclic alkyl carbamate types such as cyclopentyloxycarbonyl and adamantyloxycarbonyl; (5) alkyl types such as triphenylmethyl and benzyl; (6) trialkylsilane such as trimethylsilane; (7) thiol containing types such as phenylthiocarbonyl and dithiasuccinoyl; and (8) alkyl types such as triphenylmethyl, methyl, and benzyl; and substituted alkyl types such as 2,2,2-trichloroethyl, 2-phenylethyl, and t-butyl; and trialkylsilane types such as trimethylsilane.

The term “deprotecting agent” as used herein refers to a reagent or reagent system (reagent(s), and solvent) useful for removing a protecting group. Deprotecting agents can be acids, bases or reducing agents. For example, removal of the benzyl (Bn) group can be accomplished by reduction (hydrogenolysis), while removal of carbamates (e.g., Boc group) can be effected by use of acids (e.g., HCl, TFA, H₂SO₄, etc.), and while removal of silyl groups can be effected by use of weak acids or halides (e.g., fluoride such as provided by tetra-n-butylammonium fluoride (TBAF)), optionally with mild heating.

As used herein, the phrase “reducing agent” refers generically to any species capable of reducing another species while itself being oxidized. As used herein, the phrase “oxidizing agent” or “oxidant” refers generically to any species capable of oxidizing another species while itself being reduced.

As used herein, the term “triflating reagent” refers to a compound that is useful in a reaction in which a triflate group is attached to a hydroxy group to form a triflate ester. The triflating agent is the source of the trifluoroacetyl group. Triflating reagents include, but are not limited to, trialkylsilyl triflates, trialkylstannyl triflates, triflic anhydride (trifluoromethanesulfonic anhydride), N-phenyl-bis(trifluoromethanesulfonimide) (PhNTf₂), N-(5-chloro-2-pyridyl)triflimide, and N-(2-pyridyl)triflimide.

An “acrylonitrile derivative,” as used herein, is a compound that is derived from acrylonitrile, which has the formula CH₂CHCN, where one or more of the hydrogen atoms have been replaced by another atom or group. An example of an acrylonitrile derivative is 2-chloroacrylonitrile, where one of the hydrogen atoms of acrylonitrile has been replaced by a chlorine atom.

As used herein, the term “dilute,” when used with regard to an acid solution, refers to a solution having an acid concentration of less than about 0.1 N.

The terms “hydrogen” and “H” are used interchangeably herein.

The terms “halogen” or “halo” refer to fluorine (F), chlorine (Cl), bromine (Br), or iodine (I).

As used herein, the term “alkyl” refers to a hydrocarbon chain that can be a straight chain or branched chain, containing the indicated number of carbon atoms. For example, C₁₋₆ indicates that the group can have from 1 to 6 (inclusive) carbon atoms in it. Examples include methyl, ethyl, iso-propyl, tert-butyl, and n-hexyl.

As used herein, the term “alkylamine” refers to an amine that contains one or more alkyl groups. An alkylamine can be a primary amine, a secondary amine or a tertiary amine. For example, a secondary alkylamine is an amine that contains two alkyl groups. An example includes diisopropylethylamine.

A salt can form from a compound in any manner familiar to the skilled artisan. Accordingly, the recitation “to form a compound or salt thereof” includes embodiments where a compound is formed and the salt is subsequently formed from the compound in a manner familiar to the skilled artisan.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

All combinations of the embodiments pertaining to the aspects described herein are specifically embraced by the present invention just as if each and every combination was individually explicitly recited, to the extent that such combinations embrace possible aspects. In addition, all sub-combinations of the embodiments contained within the aspects described herein, as well as all sub-combinations of the embodiments contained within all other aspects described herein, are also specifically embraced by the present invention just as if each and every sub-combination of all embodiments are explicitly recited herein.

### Crystallization Methods

Disclosed herein are methods of converting selpercatinib Form A to selpercatinib Form B. While selpercatinib Form A can be converted into Form B using a variety of different methods, disclosed herein are crystallization-based methods that convert selpercatinib Form A to selpercatinib Form B.

Suitable methods for converting Form A to Form B include cooling crystallization, evaporation crystallization, vapor diffusion, crystallizations using one or more antisolvents (including reverse antisolvent addition), and slurry crystallization. These methods are discussed herein.

In one aspect, disclosed herein is a method of converting selpercatinib Form A to selpercatinib Form B.

In another aspect, disclosed herein is a method of converting selpercatinib Form A to selpercatinib Form B, the method comprising: combining selpercatinib Form A with a C₁-C₅ alcohol to generate a slurry and isolating selpercatinib Form B from the slurry.

In yet another aspect, disclosed herein is a method for converting selpercatinib Form A to selpercatinib Form B, the method comprising:
a. dissolving the selpercatinib Form A in a solvent comprising DMSO to form a solution;
b. adding water to the solution and thereby forming a slurry;
c. isolating the selpercatinib Form B.

In another aspect, disclosed herein is a method for converting selpercatinib Form A to form B, the method comprising: combining selpercatinib Form A and methanol to form a slurry, and stirring the slurry until >99 wt% of the Form A is converted to Form B.

Form A has unique XRPD peaks at 4.9, 9.7, and 15.5° 2θ, while Form B has unique XRPD peaks at 7.5, 10.9, and 12.0° 2θ. The 2θ values and/or peak intensities of other peaks also differ between the two forms, as may be seen in Table 1 below. To be clear, all XRPD peaks disclosed herein are ± 0.2° 2θ, unless expressly identified otherwise.

TABLE 1

X-ray Powder Diffraction Peak Analysis, Form A and Form B
FORM A

| Peak position | Relative intensity |
|---|---|
| 4.9 | 100.0% |
| 8.1 | 6.2% |
| 9.7 | 41.6% |
| 12.7 | 2.2% |
| 13.8 | 1.4% |
| 14.8 | 16.0% |
| 15.5 | 15.5% |
| 16.5 | 18.0% |
| 16.8 | 16.5% |
| 18.0 | 23.9% |
| 18.5 | 17.2% |
| 18.8 | 24.3% |
| 20.2 | 4.0% |
| 21.0 | 5.7% |
| 21.9 | 6.4% |
| 22.6 | 8.1% |
| 23.6 | 9.1% |
| 25.1 | 7.7% |
| 25.5 | 14.4% |
| 26.0 | 8.9% |
| 26.4 | 6.3% |
| 27.2 | 4.6% |
| 28.2 | 5.6% |
| 28.8 | 3.1% |
| 29.3 | 1.6% |
| 31.5 | 1.5% |
| 32.2 | 1.4% |
| 33.2 | 1.0% |
| 33.7 | 1.4% |
| Form B | |
| Peak position | Relastive intensity |
| 7.5 | 18.2% |
| 9.2 | 3.8% |
| 10.9 | 4.6% |
| 12.0 | 20.3% |
| 13.2 | 21.9% |
| 14.3 | 2.7% |
| 15.0 | 1.3% |
| 16.2 | 5.3% |
| 17.1 | 44.4% |
| 17.7 | 19.4% |
| 18.1 | 6.5% |
| 18.6 | 1.8% |
| 19.6 | 13.5% |
| 19.8 | 18.8% |
| 20.1 | 6.4% |
| 21.1 | 100.0% |
| 22.5 | 7.6% |
| 24.3 | 3.1% |
| 24.6 | 5.8% |
| 25.0 | 6.1% |
| 26.5 | 2.0% |
| 26.7 | 3.1% |
| 27.7 | 2.0% |
| 28.0 | 2.1% |
| 28.4 | 3.2% |
| 28.97 | 4.1% |
| 29.2 | 7.5% |
| 30.0 | 7.7% |
| 30.3 | 3.3% |
| 32.6 | 1.4% |
| 33.2 | 2.7% |
| 34.1 | 1.3% |
| 34.3 | 1.3% |
| 35.3 | 1.0% |

In Table 1, all peaks having relative intensities of less than 1.00 are not listed.

The XRPD patterns of Form A and Form B (above) were obtained on a Bruker D4 Endeavor X-ray powder diffractometer, equipped with a CuKα source (λ = 1.54180 Å) and a Vantec detector, operating at 35 kV and 50 mA. The sample is scanned between 4 and 40 2θ°, with a step size of 0.008 2θ° and a scan rate of 0.5 seconds/step, and using 1.0 mm divergence, 6.6 mm fixed anti-scatter, and 11.3 mm detector slits. The dry powder is packed on a quartz sample holder and a smooth surface is obtained using a glass slide. The crystal form diffraction patterns are collected at ambient temperature and relative humidity. Crystal peak positions are determined in MDI-Jade after whole pattern shifting based on an internal NIST 675 standard with peaks at 8.853 and 26.774 2θ°. It is well known in the crystallography art that, for any given crystal form, the relative intensities of the diffraction peaks may vary due to preferred orientation resulting from factors such as crystal morphology and habit. Where the effects of preferred orientation are present, peak intensities are altered, but the characteristic peak positions of the polymorph are unchanged. See, e.g. The United States Pharmacopeia #23, National Formulary #18, pages 1843-1844, 1995. Furthermore, it is also well known in the crystallography art that for any given crystal form the angular peak positions may vary slightly. For example, peak positions can shift due to a variation in the temperature at which a sample is analyzed, sample displacement, or the presence or absence of an internal standard. In the present case, a peak position variability of ± 0.2 2θ° is presumed to take into account these potential variations without hindering the unequivocal identification of the indicated crystal form. Confirmation of a crystal form may be made based on any unique combination of distinguishing peaks.

DSC-TGA analyses of an anhydrous, crystalline Form A demonstrated a melting onset of 207.6° C. and exhibited two endotherms, where the first endotherm corresponds to the melt of Form A followed by the exothermic recrystallization of Form B and then the melt of Form B. DSC-TGA analyses of an anhydrous, crystalline Form B demonstrated a single endotherm with a melting onset of 213.3° C.

While Forms A and B are anhydrous polymorphs, Form A is slightly more hygroscopic than Form B.

Forms A and B have similar solubilities. Both exhibit poor 25° C. solubility in many organic solvents, including methyl ethyl ketone (MEK), acetone, and many alcohol based solvents, while having moderate solubility (3-30 mg/ml) in dichloromethane (DCM), dimethylsulfoxide (DMSO) and THF. Form B has almost no solubility in anisole.

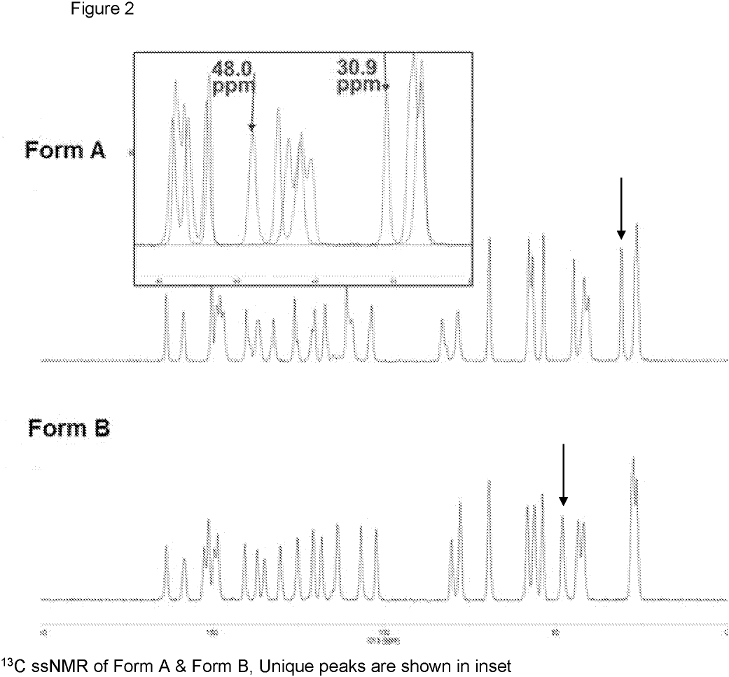

The ¹³C solid state NMR spectra of Forms A and B appear in FIG. 2. FIG. 2 also contains an overlay of a portion of the spectra, which shows Form A has a peak at 30.9 ppm that is not observable in Form B, while Form B has a peak at 48.0 ppm that is not observable in Form A. Both spectra were referenced to the high field resonance of adamantane (δ = 29.5 ppm).

¹³C Cross polarization/magic angle spinning NMR (solid-state NMR or ssNMR) spectra referenced above were obtained using a Bruker Avance III HD 400 MHz wide-bore NMR spectrometer operating at a carbon frequency of 100.62 MHz and proton frequency of 400.13 MHz, and equipped with a Bruker 4 mm double resonance probe. TOSS sideband suppression was used along with cross polarization employing SPINAL64 decoupling and a RAMP 100 shaped H-nucleus CP pulse. Acquisition parameters were as follows: 4.0 µs proton pulse, 1.5 ms contact time, 5 kHz MAS frequency, 30.2 kHz spectral width, and 34 ms acquisition time. A 3 second recycle delay is used and the number of scans is 2655. Chemical shifts are referenced to adamantane (δ = 29.5 ppm) in a separate experiment. Representative ¹³C ssNMR resonances for Form B include: 26.44, 27.37, 28.00, 41.98, 43.43, 43.91, 48.04, 53.92, 56.31, 58.32, 69.48, 77.90, 80.38, 102.32, 106.77, 113.58, 115.24, 118.23, 120.76, 125.23, 130.23, 134.86, 136.93, 140.59, 148.42, 149.50, 151.20, 152.45, 158.22, and 163.52 ppm.

The above data establishes Forms A and B: 1) have some different properties, 2) can readily be identified, and 3) Form A can convert into Form B.

A variety of different solvents can be used to convert Form A to Form B. Solvents that can be used to convert Form A to Form B include, but are not limited to C₁-C₅ alcohols (such as methanol or ethanol), water, acetonitrile (ACN, methyl tert-butyl ether (MTBE), heptane, n-butyl acetate (n-BuOAC), 81% ACN-MeOH (81 mL ACN combined with 19 mL MeOH), wet ethyl acetate, cyclopentyl methyl ether (CPME), 1,2-dimethoxyethane, ethyl acetate, ethyl formate, methyl isobutyl ketone (MIBK), nitromethane, n-propyl acetate (NPA), 1-pentanol, toluene, 1:1 MeOH:water, 1:1 EtOH:water, ACN:water, DMSO/heptane mixtures, or DMSO/water mixtures. In some embodiments, the solvents include C₁-C₅ alcohols, water, DMSO, MTBE, ACN and mixtures of two or more thereof. In still other embodiments, the solvent comprises methanol, ethanol, water, DMSO, MTBE, ACN or mixtures of two or more thereof.

As noted above, selpercatinib can form solvates; and it can also form metastable solid forms, both of which are generally not stable on drying. Observed solvates include the acetone solvate, chloroform solvate, 1,4-dioxane solvate, methyl ethyl ketone (MEK) solvate, dichloromethane (DCM) solvate, 2-butanol solvate, 1-butanol solvate, ethanol solvate, dimethylsulfoxide (DMSO)-water solvate, DMSO solvate, and the tetrahydrofuran (THF) solvate. The solvates and metastable forms usually revert to Form A during isolation and/or drying, although films or amorphous material occasionally form. The chloroform and 1,4-dioxane solvates were stable upon isolation/drying.

The Form A used in the methods described herein may contain some Form B. The amount of Form B, if present, ranges from at least about 0.1 wt% to no more than about 25 wt%, or about 0.5 wt% to about 17 wt%, or about 1 wt% to about 16 wt%.

Some non-limiting methods of converting Form A to Form B are described below.

### Conversion Method 1

In a preferred embodiment, the method comprises combining selpercatinib Form A with a solvent, such as a C₁-C₅ alcohol, to generate a slurry and isolating selpercatinib Form B from the slurry. As the slurry stirs or is otherwise agitated, selpercatinib Form B is formed. In some embodiments, the alcohol is maintained at ambient temperature. In other embodiments, the slurry is heated, which increases the rate of Form B formation. Besides the temperature differences, these two embodiments are similar, and are described below.

### Solvents

Examples of C₁-C₅ alcohols include methanol, ethanol, isopropanol, propanol, butanol, 2-butanol, 3-butanol, and 1-pentanol. In some embodiments, methanol is a preferred C₁-C₅ alcohol.

Examples of C₁-C₅ alcohols include methanol, ethanol, isopropanol, propanol, butanol, 2-butanol, and 3-butanol. In certain embodiments, the alcohol comprises methanol and/or ethanol. In an embodiment, the alcohol comprises methanol.

Aqueous alcohols may also be used, where the amount of water present is from about 0.1 wt% up to about 70 wt%, or about 1 wt% to about 50 wt%, or about 2 wt% to about 30 wt%. In an alternate embodiment, the amount of water present is about 0.5 wt% to about 20 wt% or about 1 wt% to about 15 wt%, or about 2 wt% to about 12 wt%, or about 10 wt% or less than 10 wt%. In one embodiment the alcohol comprises at least 90 wt% methanol. In another embodiment, the alcohol comprises about 90 wt% methanol and about 10 wt% water. In another embodiment, the alcohol comprises at least 95 wt% methanol and about 5 wt% water. Other solvents may be present in the alcohol mixture. In some embodiments, up to about 3 wt% of one or more other solvents may be present.

### Temperature

Temperature affects the rate at which the Form A is converted to Form B, with lower temperatures taking longer than higher temperatures. While it is possible to stir the Form A and solvent slurry at a temperature below ambient temperature, this will prolong the Form A to Form B conversion and thus, is generally avoided.

The temperature of the alcohol, such as the C₁-C₅ alcohol, is about 10-80° C., or about 20-60° C., or about 55° C. The C₁-C₅ alcohol may be at the desired temperature before the Form A material is added, or the temperature may be adjusted after the Form A material is added.

In other embodiments, the temperature of the alcohol, such as the C₁-C₅ alcohol, is 10-30° C., or about 15-25° C., or about 20° C. In other embodiment, the temperature is ambient temperature, which is the outside temperature. While Form A will convert to Form B upon stirring in a room temperature solvent, such as methanol, the conversion is faster if the Form A and solvent mixture is heated.

If the slurry is heated to such an extent that all of the Form A dissolves, the resulting solution may be filtered to remove any insoluble materials. After stirring, the solution would be stirred and cooled as detailed below.

### Time

The slurry is stirred or otherwise agitated for at least about 5 minutes or at least about 10 minutes. In some embodiments, the slurry is usually not stirred or otherwise agitated for more than 72 hours, but if desired, the slurry can be stirred or otherwise agitated for more than 72 hours. In some embodiments, the slurry is stirred for about 1-12 hours.

### Cooling

If the Form A and alcohol mixture was heated for the time indicated above, the heating is stopped and the slurry is allowed to cool for about 4 to 24 hours or about 6-18 hours, or about 12 hours.

### Isolating Form B

The Form B material may be isolated using any method known in the art. In an embodiment, the separation comprises gravity filtration. In another embodiment, the separation comprises vacuum filtration. In still another embodiment, the separation comprises the use of a centrifuge.

Fresh solvents, such as ethanol, methanol, ACN, MTBE, water or combinations of two or more thereof, can be used to wash the Form B material. More preferably, methanol, ACN, MTBE, water or combinations of two or more thereof, are used to wash the Form B material. Still more preferably, a solvent comprising methanol is used. The fresh solvent may be cooled to a temperature of about 0° C. to less than about 20° C., before it is used to wash the Form B material.

The isolated selpercatinib Form B may be dried using methods known in the art. Typical methods include heating, passing an inert gas over the solid and/or the use of pressures less than atmospheric pressure.

In a further embodiment of this example, a C₁-C₅ alcohol and selpercatinib Form A are combined and the resulting slurry is stirred or otherwise agitated for a length of time sufficient to convert the Form A to Form B. Typical stirring times are at least about 10 minutes up to about 36 hours, or about 24 hour, but typically at least about 30 minutes, or at least about 1 hour, or at least about 4 hours, or at least about 6 hours, or at least about 8 hours, or at least about 12 hours. If desired, stirring and/or agitating the mixture may go longer than 24 hours. Heating the mixture will increase the rate of conversion of the Form A to Form B.

In another embodiment of this method, the method comprises: combining selpercatinib Form A and methanol to form a slurry, and stirring the slurry until>95 wt%, >96 wt%, >97 wt%, >98 wt% or >99 wt% of the Form A is converted to Form B. The slurry is stirred for about 12 to 48 hours or about 18-24 hours. The concentration of the selpercatinib Form A in the methanol is about 6-14 mL/g or about 8-12 mL/g. In some methods, it is about 8 mL/g.

### Conversion Method 2

In another embodiment, the method comprises combining selpercatinib Form A with a solvent and the resulting mixture is heated and stirred until the Form A dissolves in the solvent. Once a solution is formed, the mixture may be filtered, if any insoluble impurities are to be removed. The mixture is then cooled and water is added. Seed crystals, if they are being used, may be added at this time. After stirring, additional water is slowly added. The mixture is then cooled to room temperature. After cooling to room temperature, the mixture is stirred, and then the Form B material is isolated

### Solvents

A variety of different solvents may be used. Importantly, the solvent should not form selpercatinib solvate; rather, it should afford the desired Form B. Examples of suitable solvents include, but are not limited to DMSO, C₁-C₅ alcohols, ACN, MTBE, water or combinations of two or more thereof. Preferred C₁-C₅ alcohols include ethanol and/or methanol. In some embodiments, DMSO is a preferred solvent. In some embodiments, the solvent contains at least 2 wt% water.

The amount of solvent used depends on the solvent that is used. Typically, 1 g of Form A is dissolved in about 8-20 mL, or about 10-15 mL, or about 11-14 mL or about 12-13 mL of solvent/ used. In some embodiments, 1 gram of Form A is dissolved in 10-15 mL/g of DMSO or 1 gram of Form A is dissolved in about 12-13 mL/g of DMSO.

### Temperature

Temperature affects the rate at which the Form A is converted to Form B, with lower temperatures taking longer than higher temperatures.

The mixture comprising Form A and the solvent is heated to a temperature that is anywhere from about 30° C. up to the boiling point of the solvent. Typically the mixture is heated to a temperature of about 50-110° C. or about 50° C. to about 70° C. In some embodiments, the mixture may be heated to about 50° C., about 60° C., about 70° C., about 80° C., about 90° C., about 100° C., or about 110° C. After the mixture is heated to the desired temperature and the Form A material is dissolved, the temperature of the solution is reduced by about 15-35° C. The temperature may be reduced by about 15° C., about 20° C., about 25° C., about 30° C., or about 35° C. In an embodiment, the solution is cooled to a temperature less than about 70° C. and greater than about 20° C.

In some embodiments, the solvent comprises DMSO and it is heated to about 50° C. to about 70° C. In a further embodiment, the DMSO is then cooled to about 50° C.

In alternate embodiments, the solvent is not heated, i.e., it is allowed to stir at ambient temperature. In these embodiments, the conversion of Form A to form takes longer.

### First Tranche of Water

When the first tranche of water is added to the solution, about 0.1-1.0 mL/g, or about 0.2-0.6 mL/g, or about 0.3 mL/g, of Form A is added (mL of water to g of Form A). In some embodiments, the first tranche of water is about 0.1 mL/g or about 0.2 mL/g, about 0.3 mL/g, about 0.4 mL/g, about 0.5 mL/g or about 0.6 mL/g.

The first tranche of water is added over about 30 seconds to about 15 minutes or about 1-10 minutes or about 4-6 minutes or about 5 minutes. Longer times may be utilized, if desired.

### Seed Crystals

If Form B seed crystals are being added to the mixture, then about 0.1-15 wt% or about 1 to about 10 wt% or about 5 wt% of Form B seed crystals are used.

In some embodiments, about 1 wt%, 2 wt%, about 3 wt%, about 4 wt%, about 5 wt%, about 6 wt%, about 7 wt%, about 8 wt%, about 9 wt%, about 10 wt%, about 11 wt%, about 12 wt%, about 13 wt%, about 14 wt%, or about 15 wt% of seed crystal is added.

The seed crystals can be prepared using the methods described herein.

### Time

After the mixture is heated and the Form A material is dissolved and the mixture temperature was reduced by out 50-110° C., and the seed crystals were added - if seed crystals were used, then the mixture is stirred for about 1-96 hours, or about 6-72 hours, or about 8-24 hours. In some embodiments, the mixture is stirred for at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, or at least 24 hours.

### Second Tranche of Water

After stirring for the 1-96 hours, a second tranche of water is slowly added. The amount of water in the second tranche is about 0.3-6 mL/g, 0.50-3.0 mL/g (mL of water per gram of Form A), about 0.75-1.5 mL/g, or about 0.9-1.20 mL/g. In some embodiments, the second tranche of water is about 0.90 mL/g, about 0.91 mL/g, about 0.92 mL/g, about 0.93 mL/g, about 0.94 mL/g, about 0.95 mL/g, about 0.96, mL/g about 0.97 mL/g, about 0.98 mL/g, about 0.99 mL/g, about 1.00 mL/g, about 1.01 mL/g, about 1.02 mL/g, about 1.03 mL/g, about 1.04 mL/g, about 1.05 mL/g, about 1.06 mL/g, about 1.07 mL/g, about 1.08 mL/g, about 1.09 mL/g, about 1.10 mL/g, about 1.11 mL/g, about 1.12 mL/g, about 1.13 mL/g, about 1.14 mL/g, about 1.15 mL/g, about 1.16 mL/g, about 1.17 mL/g, about 1.18 mL/g, about 1.19 mL/g, about 1.20 mL/g of Form A.

The second tranche of water is added slowly, i.e., it takes about 0.5-24 hours or about 1-12 hours to add the entire second tranche of water. In some embodiments, it takes about 1 hour, about 2 hours, about 3 hours, about 4 hours, about 5 hours, about 6 hours, about 7 hours, about 8 hours, about 9 hours, about 10 hours, about 11 hours, or about 12 hours to add the entire second tranche of water.

### Cooling

After the second tranche of water is added, the mixture is cooled by about 15-30° C. to a temperature of about 20-30° C. In some embodiments, the mixture is cooled to about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., or about 30° C. In one embodiment, the final temperature, after cooling is room temperature. In other embodiments, the mixture is cooled to a temperature of about 30-55° C. In these embodiments, the yield tends to be slightly lower than when lower temperatures are used.

After the second tranche of water is added, the mixture is cooled at a rate of about 1 20° C./hr, or about 3-17° C./hr, or about 5-15° C./hr, until the desired temperature is reached. In one embodiment, the rate of cooling is about 1° C./hr, about 2° C./hr, about 3° C./hr, about 4° C./hr, about 5° C./hr, about 6° C./hr, about 7° C./hr, about 8° C./hr, about 9° C./hr, about 10° C./hr, about 11° C./hr, about 12° C./hr, about 13° C./hr, about 14° C./hr, about 15° C./hr, about 16° C./hr, about 17° C./hr, about 18° C./hr, about 19° C./hr, or about 20° C./hr.

After the desired temperature is reached, the mixture is stirred for about 1 to about 72 hours or about 2 to 48 hours. In some embodiments, the mixture is stirred for at least two hours. In other embodiments, the mixture is stirred for less than 72 hours.

### Isolating Form B

### Form B is Isolated as Described Above

Fresh solvents, such as ethanol, methanol, ACN, MTBE, water or combinations of two or more thereof, can be used to wash the Form B material. More preferably, methanol, ACN, MTBE, water or combinations of two or more thereof, are used to wash the Form B material. Still more preferably, a solvent comprising methanol is used. The fresh solvent may be cooled to a temperature of about 0° C. to less than about 20° C., before it is used to wash the Form B material.

In embodiments where the solvent comprises DMSO, the isolated selpercatinib Form B is washed with methanol until the isolated selpercatinib Form B contains less than 0.5 wt % DMSO.

In a further example of this method, the selpercatinib Form A is dissolved in a room temperature solvent comprising DMSO to form a solution having a concentration of about 10-15 mL/g of DMSO per gram of Form A. Then water is added. The mixture is then allowed to rest, during which time Form B will form. The Form B can then be isolated or additional water can be added and after further stirring (as described above) the Form B can be isolated.

In another example of this method, the selpercatinib Form A is dissolved in DMSO at about 60-80° C. or about 70° C. to form a solution having a concentration of about 10-15 mL/g of DMSO per gram of Form A; cooling the mixture to about 40-60° C. or about 50° C.; adding water; seeding the resulting mixture with Form B seed crystals, stirring the mixture, adding more water, heating the mixture; cooling the mixture and isolating the Form B. The initial amount of water added is about 0.1 mL/g of Form A to about 0.5 mL/g of Form A, or about 0.3 mL/g of Form A. The amount of seed crystals that may be used ranges from about 1-10 wt%, or about 5 wt%, based on the amount of Form A. The seed crystal containing mixture is stirred for about 8-24 hours or about 12 hours. The second addition/tranche of water is about 1.0-1.5 mL/g of Form A, or about 1.10-1.15 mL/g or about 1.14 mL/g. The second addition/tranche of water is added over about 3-8 or about 5 hours. After the second addition/tranche of water is added, the slurry is cooled to about 20-30° C. or about 25° C. The rate of cooling the slurry from about 70° C. to about 25° C. is about 10° C./hour, until about 25° C. is reached. The about 25° C. slurry is stirred for at least about 2 hours, and then it is heated to about 60-80° C. or 70-75° C. or about 73° C. and stirred for about an hour. The slurry is then cooled again to about 20-30° C. or about 25° C. The slurry is cooled from about 73° C. to about 25° C. at a rate of about 10° C./hour. After stirring for at least about 30 minutes to about 8 hours, or about 1-8 hours or about 2 hours, the selpercatinib Form B is isolated, for example, by filtration.

Crystallization methods effective for the conversion of Form A to Form B are further exemplified in the illustrative embodiments described in the Examples.

### Direct Synthesis of Form B of the Compound of Formula I

In another aspect, the disclosure relates to a process for preparing a compound of Formula I (i.e., selpercatinib)

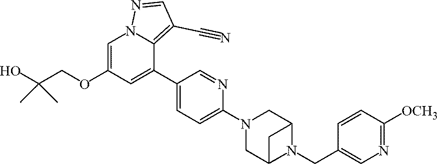 [SMILES: COc1ccc(CN2C3CC2CN(c2ccc(-c4cc(OCC(C)(C)O)cn5ncc(C#N)c45)cn2)C3)cn1]

as Form B, or a pharmaceutically acceptable salt thereof.

In embodiments, the process for preparing selpercatinib Form B comprises synthesis of one or more precursor compounds via synthetic methods such as those disclosed and described elsewhere (e.g., U.S. Pat. 10,112,942, incorporated herein by reference in its entirety). Illustrative Schemes 1 and 2 below show general methods for preparing selpercatinib Form B, as well key intermediate compound [3], from precursor compound [2]:

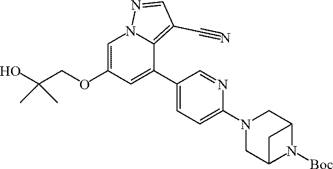 [SMILES: CC(C)(O)COc1cc(-c2ccc(N3CC4CC(C3)N4C(=O)OC(C)(C)C)nc2)c2c(C#N)cnn2c1]

A detailed description of synthetic methods that can provide precursor compound [2] (tert-butyl 3-(5-(3-cyano-6-(2-hydroxy-2-methyl-propoxy)pyrazolo[1,5-a]pyridine-4-yl)pyridine-2-yl)-3,6-diazabicyclo[3.1.1]heptane-6-carboxylate) are disclosed, for example, in U.S. Pat. 10,745,419 and 10,112,942, and International Patent Publication WO 2018/071447 each of which are incorporated by reference in their entirety herein. In brief overview of one non-limiting embodiment, compound [2] may be prepared by reaction of 4-(6-fluoropyridin-3-yl)-6-(2-hydroxy-2-methylpropoxy)pyrazolo[1,5-a]pyridine-3carbonitrile; 3,6-diaza-bicyclo[3.1.1]heptane-6-carboxylic acid tert-butyl ester and K₂CO₃₍ₛ₎ (at 1:1:6.67 molar eqs.) in DMSO, with stirring under heat (e.g., 12 hr at 90° C.). The resulting thick slurry is diluted with additional DMSO and is stirred under heat (e.g., an additional 12 hr at 90° C.). After reaction the mixture is cooled to ambient temperature and is diluted with water, and the resulting aqueous mixture is washed with dichloromethane. The combined organic extracts are dried over anhydrous MgSO₄₍ₛ₎, filtered, and concentrated in vacuo. The resulting residue is purified by silica chromatography (EtOAc/hexanes as the gradient eluent system) to provide compound [2] in high yield. Those skilled in the art will appreciate that other synthetic routes can be used to synthesize compound [2]. Those skilled in the art will further appreciate that compounds [2] may comprise amine protecting groups other than Boc, including the non-limiting examples of formyl, acetyl, trifluoroacetyl, benzyl, benzoyl, carbamate, benzyloxycarbonyl, p-methoxybenzyl carbonyl, trimethylsilyl, 2-trimethylsilyl-ethanesulfonyl, trityl and substituted trityl groups, allyloxycarbonyl, 9-fluorenylmethyloxycarbonyl, nitroveratryloxycarbonyl, p-methoxybenzyl and tosyl. In some embodiments, the protecting group is tert-butyloxycarbonyl (Boc).

Generally, methods for direct synthesis of Form B selpercatinib in accordance with the disclosure comprise reacting compound [2] (tert-butyl 3-(5-(3-cyano-6-(2-hydroxy-2-methyl-propoxy)pyrazolo[1,5-a]pyridine-4-yl)pyridine-2-yl)-3,6-diazabicyclo[3.1.1]heptane-6-carboxylate) under conditions that are effective (1) to remove the protecting group (e.g., Boc, as shown in [2]) and (2) for the silylation of the hydroxyl group on the 2-hydroxy-2-methyl-propoxy substituent group (e.g., TMS, as shown in [3]). The silylated and deprotected compound [3] is then reacted in an organic solvent (e.g., anisole) with 6-methoxy-3-pyridinecarboxaldehyde in the presence of a reducing agent and an acid.

The silyl moiety (e.g., TMS in some illustrative embodiments) is removed under conditions effective for deprotecting such as, for example, addition of a fluoride source (e.g., tetrabutylammonium fluoride (TBAF)). After reaction and removal of the silyl protecting group, the pH of the reaction mixture is adjusted with a base and is cooled to allow formation and isolation of crystalline Form B selpercatinib.

In some embodiments the conditions effective to remove the protecting group and for the silylation may comprise a solvent selected from polar organic solvents, such as alcohols (e.g., MeOH, EtOH), organic acids (e.g., aryl sulfonic acids such as p-toluenesulfonic acid), aprotic solvents (e.g., acetonitrile), acyl halides in alcohols (e.g., acetyl chloride in methanol to generate an HCl solution), esters (e.g., ethyl acetate), ethers (e.g., anisole), and combinations thereof. In some embodiments, the reaction comprises a deprotecting agent that may comprise trifluoroacetic acid, hydrochloric acid, hydrobromic acid, hydriodic acid, phosphoric acid, sulfuric acid, methanesulfonic acid, p-toluene sulfonic acid, acetyl chloride, aluminum trichloride, and boron trifluoride. In some embodiments, the deprotecting agent is sulfuric acid, acetyl chloride, or p-toluene sulfonic acid. In some embodiments the conditions may comprise heating the reaction mixture, optionally to reflux, for a period of time ranging from about 1 hr to about 8 hrs or longer (e.g., overnight, or about 12 hrs).

In some embodiments the silyl group used in the reaction may comprise trimethylsilyl (TMS), triethylsilyl (TES), tert-butyldiphenylsilyl (TBDPS), isopropyldimethylsilyl (IPDMS), diethylisopropylsilyl (DEIPS), tert-butyldimethylsilyl (TBS/TBDMS), tetraisopropyldisiloxanylidene (TIPDS), di-t-butylsilylene (DTBS), or triisopropylsilyl (TIPS). The presence of the silyl group (e.g., TMS group on compound [3]), in addition to acting as a protecting group, provides added solubility of the compound in the solvent anisole, which can be considered an antisolvent for selpercatinib Form B, compound [2], and the non-silylated derivative of compound [3].

The silyl group can be added using methods known in the art.

In some embodiments, the reaction of compound [3] with 6-methoxy-3-pyridinecarboxaldehyde is performed with anisole as the solvent, given that compound [3] demonstrates greater solubility in anisole than the 2-hydroxy-2-methyl-propoxy form of [3]. In some embodiments the reducing agent in the reaction may comprise an alkali metal borohydride, a hydrazine compound, citric acid, a citric acid salt, succinic acid, a succinic acid salt, ascorbic acid, and an ascorbic acid salt. In some embodiments, the reducing agent is selected from a sodium borohydride, a lithium borohydride, a nickel borohydride, and a potassium borohydride. In some embodiments, the lithium borohydride is selected from lithium borohydride and lithium triethylborohydride. In some embodiments, the sodium borohydride is selected from sodium triacetoxy borohydride (STAB), sodium borohydride, and sodium cyanoborohydride. In some embodiments, the reducing agent is STAB. In some embodiments, the acid in the reaction acts as a catalyst for reaction and may comprise an inorganic acid (e.g., HCl, H₂SO₄, etc.), or an organic acid having solubility in water (e.g., acetic acid, pivalic acid, etc.). In some embodiments, the acid comprises pivalic acid.

The resulting compound is deprotected under conditions that are adequate to remove the silyl group (e.g., TMS) but that are not so harsh as to react with and decompose reaction product (i.e., selpercatinib). In some embodiments, deprotection of the silyl group comprises adding a fluoride source (e.g., tetrabutylammonium fluoride (TBAF), pyridine·(HF)ₓ, trimethylamine trihydrofluoride (Et₃N·3HF), hydrofluoric acid, tris(dimethylamino)sulfonium difluorotrimethylsilicate (TASF), ammonium fluoride (H₄NF)) or weak acids to the reaction in amounts effective to react with the silyl group. Conditions for the deprotection step can comprise a buffered fluoride source and can be determined empirically, with the conditions being maintained sufficiently gentle to avoid decomposition reactions.

After reaction, the pH of the reaction mixture is adjusted with a base (e.g., K₂CO₃ slurry) and is cooled to allow formation and isolation of crystalline Form B selpercatinib. In some embodiments, the crystallization can further comprise addition of a small amount of seed crystals of selpercatinib Form B. In some further embodiments, the crystallization can comprise any of the crystallization techniques described herein that may be effective in converting any remaining amount of selpercatinib Form A to Form B.

Although specific starting materials and reagents are depicted in the Schemes and the related description below, other starting materials, reaction conditions, and reagents can be substituted to provide the target compound (i.e., selpercatinib Form B) in accordance with the disclosure.

In some embodiments of this aspect, the synthetic method comprises the general reaction scheme depicted in Scheme 1.

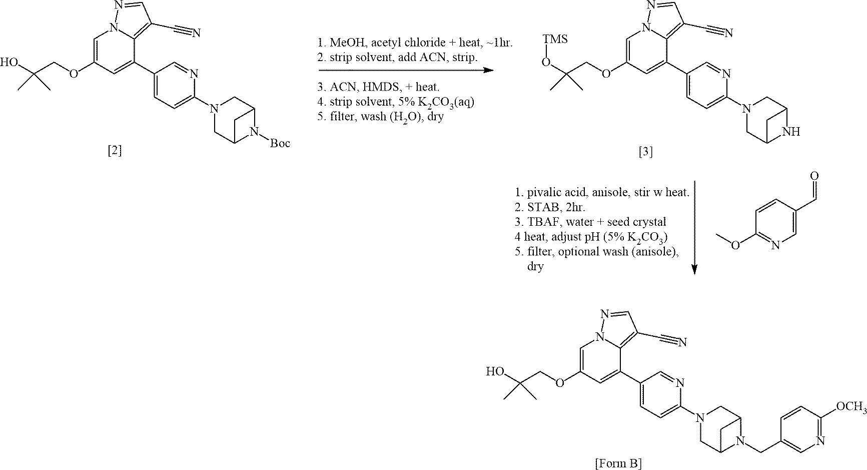

In some embodiments of this aspect, the process comprises the general reaction scheme depicted in Scheme 2.

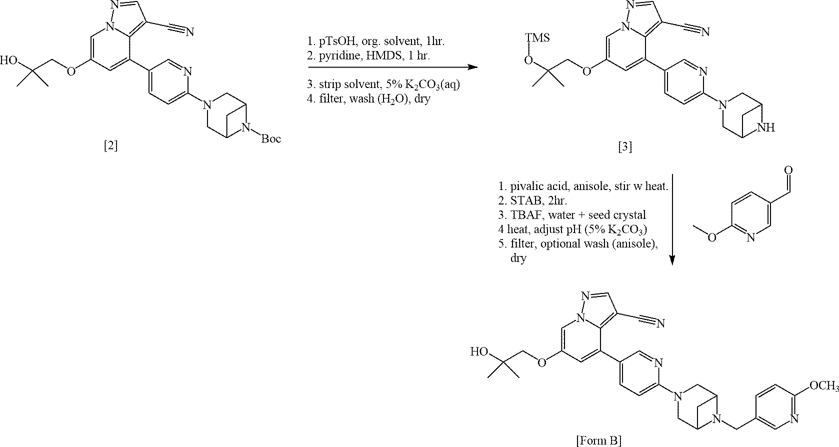

Regardless of whether selpercatinib Form B is obtained by direct synthetic method or conversion from selpercatinib (i.e., amorphous selpercatinib or selpercatinib in another polymorphic form) according to aspects and embodiments in accordance with the disclosure, it can be further provided as a pharmaceutically acceptable salt thereof, or pharmaceutical composition thereof, and can exhibit greater thermodynamic stability relative to selpercatinib in its other polymorphic and/or amorphous forms. Selpercatinib Form B retains its activity as a RET inhibitor, and can be evaluated and assessed for activity by any assays known in the art including those assays described in, e.g., PCT Publication No. WO2018/071447 and U.S. Pat. Application Publication No. US 20180134702, each of which is incorporated by reference in its entirety.

The Examples that follow are provided merely for purposes of illustrating and describing certain embodiments falling within the scope of the methods described herein, and an encompassed by the claims.

### EXAMPLES

The selpercatinib (6-(2-hydroxy-2-methylpropoxy)-4-(6-(6-((6-methoxypyridin-3-yl)methyl)-5 3,6-diazabicyclo[3.1.1]heptan-3-yl)pyridin-3-yl)pyrazolo[1,5-a]pyridine-3-carbonitrile) used in the crystallization procedures described herein was made using the techniques and methods described in U.S. Pat. No. 10,112,942.

### Example 1: Cooling Crystallization

264 mg of Form A dissolved in 20 mL DCM and dispensed in equal portions in (15) 8 mL vials. These vials were then placed in a vacuum oven at 70° C. to remove solvent. Birefringent white solid was observed in all the vials. Respective solvents (See Table 2.) were added at 50° C. with shaking. The heat source was turned off and the samples allowed to cool to room temperature (RT) naturally. The samples were stirred overnight and the resulting solids isolated by vacuum filtration followed by were air drying. Vials with no solids were placed in the refrigerator for 3 days and if no precipitation occurred, were then left to evaporate in a fume hood for 1 day. XRPD data were collected on wet solid (where possible). Approximately ⅔^{rd} (~66%) of the experiments yielded solvates which were metastable on isolation and drying. These metastable solvates (except chloroform solvate) transformed to Form A as soon as taken out of the mother liquor. 81% ACN-MeOH afforded Form B, while anisole afforded Form A.

TABLE 2

Summary of Cooling Crystallization experiments

| Solvent | Anti-solvent | Temperatur e °C | Product |
|---|---|---|---|
| DMSO | MTBE | RT | Form B |
| 4:1 toluene-DMF | MTBE | 50 | Form B |
| acetone | heptane | 50 | Form B |
| THF | heptane | 50 | Form B |
| MeOH | MTBE | 50 | Form B + Form A (minor) |
| DCM | n-BuOAc | 50 | Form A // Form B |

### Example 2: Evaporation & Vapor Diffusion Crystallization

The evaporation plate was prepared by dissolving 5 mg of Form A in 0.9-12 mL solvent into (33) vials. The evaporative solutions were manually syringe filtered into clean vials, covered with parafilm pierced with a pinhole and allowed to evaporate to dryness in the fume hood at room temperature (RT) and ambient humidity. The solutions for vapor diffusion were placed in 20 mL chambers containing 5 mL of an antisolvent and capped tightly.

Approx. half of the crystallization experiments yielded solvates or a mixture of solvates with Form A. A predominant number of solvates converted to Form A on desolvation. It is suspected that a templating effect due to structural similarity may be directing nucleation of the metastable form. Form B was obtained only from two crystallization experiments using ACN and 5:1 MeOH-THF. X-ray diffraction amorphous form/film was obtained from 5 solvent systems (THF, 11:1 IPA:acetic acid, benzyl alcohol, acetic acid and 10:1 EtOH:DMF). Chloroform and 1,4-dioxane solvates were stable on isolation and solid state characterization data were collected. IPA is isopropyl alcohol, THF is tetrahydrofuran and DMF is dimethylformam ide.

Vapor diffusion experiments afforded a variety of solvates or amorphous material. Five solvates, i.e., DCM, 1-BuOH, EtOH, THF, and DMSO were metastable and afforded Form A on isolation. A DMSO/heptane mixture afforded a mixture of Form A and Form B.

### Example 3: Antisolvent Crystallization

Antisolvent addition experiments were prepared by dissolving various amounts (9-36 mg) of Form A in 1-15 mL solvent in (29) 4 mL vials. For the first 17 vials, antisolvent was dripped into syringe filtered solutions until either precipitation occurred or the volume of antisolvent equaled or was greater than the volume of solvent. For the second 12 vials, the solution was syringe filtered into a clean vial containing 5 mL of antisolvent. Solids were isolated by vacuum filtration and air drying. Vials where no precipitation was observed, were evaporated for a period of up to 2 weeks. 71% of the antisolvent addition yielded Form A or a labile solvate leading to Form A. Form B appeared in 24% of the experiments (one result was amorphous). For reverse antisolvent addition, 83% of the experiments resulted in Form A or solvate and 17% of the experiments yielded Form B.

### Example 4: Slurry Crystallization

Slurries of Form A vials were prepared with 10 mg of Form A in 4 mL vials. Solvent was added in accordance with the solubility of Form A in these solvents to create a slurry density. The slurries were shaken for about 3 days on a 500 rpm shaker block at 22° C. Solids were analyzed as wet cakes by XRPD. The majority of the slurry screen resulted in solids consistent with Form A or solvates which transformed to Form A during isolation/drying.

Another slurry plate comprising 10 mg of Form A in 4 mL vials was prepared in a similar way as mentioned in the previous paragraph. The slurries were shaken for 24 hours on a 500 rpm shaker block at 22° C. After 24 hours, the mother liquor in each vial was replaced with fresh respective solvent. The slurries were then stirred for 15 days. Solids were analyzed wet as well as dry by XRPD. In about ⅔^{rd} (~66%) of the experiments, Form B was observed. In remaining ⅓^{rd} (~33%) of the experiments, mixture of Form A and B or Form A and solvate (1 case) were obtained. These results suggest equilibrium was not reached, which could be due to: 1) the solubility limitation of Form A in the tested solvents or 2) the minimal thermodynamic driving force for phase transformations near transition points. In the slurry crystallization experiments, anisole again afforded Form A.

TABLE 3

Summary of slurry conditions and results

| | | | |
|---|---|---|---|
| Solvent | Temp (°C) | Final Form wet (XRPD) | Final Form dry (XRPD) |
| MeOH | RT | Form B | Form B |
| EtOH | RT | Form B | Form B |
| ACN | RT | Form B | Form B |
| wet EtOAc | RT | Form B | Form B |
| nBuOAc | RT | Form A+ B | Form A + B |
| CPME | RT | Form B | Form B |
| 1,2-dimethoxyethane | RT | Form B | Form B |
| EtOAc | RT | Form B | Form B |
| ethyl formate | RT | Form B | Form B |
| Heptane | RT | Form A + B | Form A + B |
| MIBK | RT | Form B | Form B |
| Nitromethane | RT | amorphous | Form B + A (minor) |
| NPA | RT | amorphous | Form A + B (minor) |
| 1-pentanol | RT | amorphous | Form A + B |
| Toluene | RT | Form A + B | Form A + B |
| 1:1 MeOH-water | RT | Form B | Form B |
| 1:1 EtOH-water | RT | Form B | Form B |
| DMSO | RT | amorphous | Form B |
| water | RT | amorphous | Form A + B |
| methanol | RT (2 hrs) | Form B | Form B |
| Methanol:water (aw =0.5) | RT (1 day) | Form B | Form B |
| Acetonitrile:water (aw =0.8) | RT (1 day) | Form B | Form B |
| Water | RT (5 days) | Form A+ B (minor) | Form A+ B (minor) |
| Water | RT (7 days) | Form A+ B (minor) | Form A+ B (minor) |

In Table 3 above, the slurries were stirred for 15 days, unless a different time is described.

### Example 5: Solvent-Assisted Grinding

Two experiments using solvent assisted mechnaical grinding were carried out¹ . In one experiment, Form B was observed when DMSO was used as the solvent. No form change, i.e., Form A was observed when water was used as a solvent.

### Example 6: Converting Form A to Form B

Selpercatinib (2.0 g,) is suspended in methanol (200 mL) and stirred at 55° C. at 750 rpm. The suspension is stirred for 60 minutes at 55° C. Heating is stopped and the suspension is allowed to cool naturally to RT. The solid is collected by filtration and dried under vacuum for 4 hr to provide crystals of the title compound (1.72 g, 86%).

### Example 7: Converting Form A to Form B

Selpercatinib Form A (152.0 g,) is suspended in methanol (1.5 L) and stirred at RT at 750 rpm. The suspension is stirred overnight at RT (about 20° C.). The solid is collected by filtration under vacuum. The solid is dried under full house vacuum with nitrogen purge at 45° C. to provide crystals of the title compound (148.28 g, 97.6%).

### Example 8: Converting Form A to Form B

At room temperature, stir selpercatinib Form A in methanol (8 mL/g) for 18-24 hours. Filter to isolate solids. Dry the solids under vacuum at 45° C. with a slight N₂ purge.

### Example 9: Converting Form A to Form B

With stirring, Form A is dissolved in DMSO (13 mL/g) at 70° C. to obtain a clear solution. The solution is cooled to 50° C. Water is charged (0.3 mL/g), and then the solution is seeded with Form B seed crystals (5 wt %, based on the amount of form A that was used). Stir for 12 hours, then charge water (1.14 mL/g) over 5 hours. Cool the slurry to 25° C. at 10° C./h. Stir at least 2 hours. Heat the slurry to 73° C. and stir 1 hour. Cool the slurry to 25° C. at 10° C./h. Stir at least 2 hours. Isolate the solids by filtration. Wash the wet cake 3× with MeOH (8 mL/g). Dry the solids under vacuum at 45° C. with a slight N₂ purge.

### Example 10: Synthesis of Form B

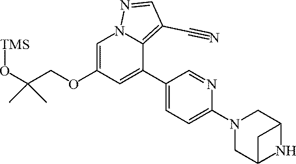 [SMILES: CC(C)(COc1cc(-c2ccc(N3CC4CC(C3)N4)nc2)c2c(C#N)cnn2c1)O[Si](C)(C)C]

4-[6-(3,6-diazabicyclo[3.1.1]heptan-3-yl)-3-pyridyl]-6-(2-methyl-2-trimethylsilyloxypropoxy)pyrazolo[1,5-a]pyridine-3-carbonitrile [3]

This synthetic route to Form B of the compound of Formula I (i.e., selpercatinib) can comprise any synthetic route that generates the compound tert-butyl-3-[5-[3-cyano-6-(2-hydroxy-2-methylpropoxy)pyrazolo[1,5-a]pyridin-4-yl]-2-pyridyl]-3,6-diazabicyclo[3.1.1]heptane-6-carboxylate [2].

Into a round bottom flask (3-necked) equipped with overhead stir, condenser, and thermocouple is added methanol (200 mL, 100%) and acetyl chloride (3.1 mL, 44 mmol, 100%). The mixture is allowed to react prior to addition of tert-butyl 3-[5-[3-cyano-6-(2-hydroxy-2-methyl-propoxy)pyrazolo[1,5-a]pyridin-4-yl]-2-pyridyl]-3,6-diazabicyclo[3.1.1]heptane-6-carboxylate [2], (9.9965 g, 19.81 mmol, 100%). Upon addition, the reaction is heated to about 60° C. (63° C.). The temperature was adjusted to reduce the amount of observable off-gassing and avoid potential overdriving of the attached condenser. After assaying the reaction to determine complete conversion (about 2 hr) the solvent was stripped. To that mixture, acetonitrile (ACN) was added (about 100 mL) rinsing down the sides of the reaction vessel. The solvent mixture was stripped again and maintained under nitrogen atmosphere.

To the reaction vessel is added additional ACN (300 mL, 100%) and hexamethyldisilazane (“HMDS” 25 mL, 119 mmol, 100%). The reaction is stirred at ambient temperature for about 1 hr. prior to initial sampling of the reaction mixture, forming the title compound at about 1.6% based on amount of [2]. The reaction was allowed to proceed overnight at ambient temperature. Following sampling of the overnight reaction, the mixture is heated to 40° C. and sampled after an hour at temperature. The heat of the reaction is raised to 56° C. During the temperature increase the mixture refluxed and foamed which is presumed to indicate evolution of ammonia. After about 1-1.25 hr. at temperature, the reaction is sampled, with continued observable reflux. The reaction is maintained at temperature for an additional 3 hr. and is again sampled. The reaction solvent was stripped and aqueous potassium carbonate solution (100 mL, 50.45 mmol, 5 mass%) is slurried into the reaction vessel. The resulting mixture is washed with water (25 mL) and dried providing 7.89 g of the title compound [3] (78% yield). (mass spec, m/z = 477.20, 477.30 (M+H). ¹H NMR (400 MHz, DMSO-d₆) d: 8.55 (s, 1H), 8.06 (d, 1H), 7.82 (dd, 1H), 7.66 (dd, 1H).

Alternative Process. Into a reaction vessel equipped with overhead stir, condenser, and thermocouple is added tert-butyl 3-[5-[3-cyano-6-(2-hydroxy-2-methyl-propoxy)pyrazolo[1,5-a]pyridin-4-yl]-2-pyridyl]-3,6-diazabicyclo[3.1.1]heptane-6-carboxylate [2], (9.9965 g, 19.81 mmol, 100%), and p-toluenesulfonic acid (2.1 eq) in 10 volumes of an organic solvent. The mixture is reacted for 1 hr, after which time is added pyridine (2.1 eq.) and hexamethyldisilazane (“HMDS” 6 eq.). This reaction mixture is stirred for about another 1 hr. to provide the title compound [3].

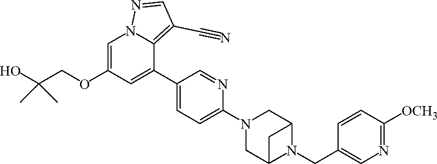 [SMILES: COc1ccc(CN2C3CC2CN(c2ccc(-c4cc(OCC(C)(C)O)cn5ncc(C#N)c45)cn2)C3)cn1]

6-(2-hydroxy-2-methylpropoxy)-4-(6-(6-((6-methoxypyridin-3-yl)methyl)-3,6-diazabicyclo[3.1.1]heptan-3-yl)pyridin-3-yl)pyrazolo[1,5-a]pyridine-3-carbonitrile

### Form B

Into a reaction vessel equipped with a magnetic stirrer is added 4-[6-(3,6-diazabicyclo[3.1.1]heptan-3-yl)-3-pyridyl]-6-(2-methyl-2-trimethylsilyloxypropoxy)pyrazolo[1,5-a]pyridine-3-carbonitrile [3] (0.9981 g, 2.094 mmol, 100 mass%), 6-methoxy-3-pyridinecarboxaldehyde (i.e., 6-methoxynicotinaldehyde, 0.4909 mg, 0.003401 mmol, 95 mass%), pivalic acid (0.5328 mg, 0.005217 mmol, 100 mass%), and anisole (10 mL, 91.8 mmol, 100 mass%), and is stirred to form a slurry. Heat is applied with stirring until a homogeneous solution mixture is obtained. The solution is cooled to ambient temperature and remains as a homogeneous solution. Once cooled, sodium triacetoxyborohydride (1.0840 g, 5.1147 mmol, 100 mass%) is added and is allowed to react. Analysis of the reaction after 2 hr. indicated formation of the TMS-protected derivative of the title compound.

After completion of the reaction, the method may is continued to remove the TMS protection and crystallize Form B. To the mixture is added water (1 mL, 55.5099 mmol, 100 mass%) and tetrabutylammonium fluoride trihydrate (0.6070 g, 2.322 mmol, 100 mass%) and, optionally, an amount (~10 mg) of seed crystals of the title compound as Form B to the mixture. If no crystals are observable after a period of time, the mixture can be warmed to 50° C. After maintaining the elevated temperature overnight the reaction is sampled, confirmed as complete, but without any observed crystallization. The pH of the mixture (slightly acidic) is adjusted by addition of potassium carbonate (5 mass% in water) as a slurry, added in 1 mL aliquots until any observed bubbling ceased and pH tested basic. The mixture is stirred overnight and sampled, providing the title compound without detectable impurities; obtained at a total isolated yield of 54%. (mass spec, m/z = 526.30 (M+H). ¹H NMR (400 MHz, DMSO-d₆) d: 8.55 (s, 1H), 8.06 (d, 1H), 7.82 (dd, 1H), 7.66 (dd, 1H)

Alternative Process. To a reaction vessel equipped with a magnetic stirrer is added 4-[6-(3,6-diazabicyclo[3.1.1]heptan-3-yl)-3-pyridyl]-6-(2-methyl-2-trimethylsilyloxy-propoxy)pyrazolo[1,5-a]pyridine-3-carbonitrile [3](1.00 g, 2.10 mmol) , 6-methoxy-3-pyridinecarboxaldehyde (1.6 eq), pivalic acid (~ 5 vol eq.)), sodium triacetoxyborohydride (2.5 eq.), and anisole (10 mL, 91.8 mmol, 100 mass%), and reacted for about 1 hr. To the reaction mixture is added water (10 mL). The mixture is filtered through celite (diatomaceous earth, filter aid). The layers are separated, with saturated sodium chloride solution (10 mL) added to the organic layer. The layers are separated. To the organic layer is added 5N HCI (1 mL). The mixture is heated to 95° C. for 3 hr. After reaction, the pH of the mixture (acidic) is adjusted by addition of potassium carbonate to a pH of 9. The mixture is cooled to allow crystallization. Resulting crystals of selpercatinib Form B are filtered, washed with methyl tert-butyl ether (MTBE) and dried to obtain pure title compound.

### Example 11

Physical and chemical stability of Form B is an important attribute not only with respect to ensuring dissolution and solubility, but also for API and dosage form drug development and manufacturing operations (drying, storage, shipping transfers, etc.). Not all crystalline forms have the needed stability to enable drug development. A crystal form that is stable with respect to both temperature and humidity is desired. In order to assess the stability of the crystal form of selpercatinib an accelerated stability study iss carried out. Samples of Form B are weighed into 20 mL scintillation vials and placed (open dish) into bell jars with saturated salt solutions in ovens at the specified temperatures and for the specified times in Table 4. The Form B is analyzed before and after the accelerated stability study and is collected on a Bruker D8 Advance XRPD, equipped with a CuKα source (wavelength = 1.54056 Å) and a Linxeye detector, and operating at 40 kV and 40 mA, with a 0.2 mm divergence slit. Each sample is scanned from 4° to 30° 2θ in 0.02° steps at a rate of 0.2 seconds per step. Assay and impurities relative to the starting material are assessed using an Agilent 1260 HPLC System with Diode Array Detector. Samples are prepared at appropriate concentrations in 50/50 0.1%TFA in water/0.1%TFA in ACN and evaluated using the following HPLC conditions: Column Zorbax Bonus-RP, 75 × 4.6 mm i.d., 3.5 micron, mobile phase A is 0.1% TFA in water, mobile phase B is 0.1% TFA in ACN, gradient is 95% A at time 0, 23% A at time 9.5-12.1 minutes, 5% A at time 13-16 minutes, 95% A at time 16.1-20 minutes with a flow rate of 1.5 mL/min, a column temperature of 30° C., UV detection wavelength of 210 nm, and injection volume of 3 µL. The stability of the Form B is characterized and found to be chemically and physically stable under the testing conditions (Table 4).

TABLE 4

Stability of the crystal form of selpercatinib

| Time Point (days) | Temp (°C) | RH (%) | Crystal Form ¹ | Assay (%) ² | Impurities (%) |
|---|---|---|---|---|---|
| 0 | - | - | Form | - | 0.09 |
| 7 | 40 | 11 | NC | 99.2 | 0.10 |
| 14 | 40 | 11 | NC | 99.9 | 0.09 |
| 7 | 40 | 75 | NC | 99.4 | 0.11 |
| 14 | 40 | 75 | NC | 99.9 | 0.10 |
| 7 | 50 | 30 | NC | 99.4 | 0.09 |
| 14 | 50 | 30 | NC | 100.5 | 0.09 |
| 7 | 50 | 50 | NC | 99.2 | 0.10 |
| 14 | 50 | 50 | NC | 100.4 | 0.11 |
| 7 | 70 | 11 | NC | 100.7 | 0.09 |
| 14 | 70 | 11 | NC | 99.3 | 0.10 |
| 7 | 70 | 75 | NC | 100.3 | 0.10 |
| 14 | 70 | 75 | NC | 100.9 | 0.11 |

¹ Note, the form is compared to the XRPD of the unstressed (time 0) sample; NC
= No Change.
² Note, the assay value is determined in comparison to the unstressed (time 0) sample.

### Example 12: Solubility

Solubility studies were completed on the crystal form of selpercatinib described herein. Aqueous media covering the physiological pH range and three simulated fluids were used in these studies. A sufficient quantity of solid compound to saturate the volume of solvent is weighed into a vessel with approximately 1 mL of the specified solvent. Samples are mixed at 37° C. in an incubator shaker set at 100 rpm. After equilibration, samples are transferred to centrifuge filters (Durapore PVDF, 0.22 µm pore size) and centrifuged for 3 min at 10,000 rpm, while maintaining 37° C. A 100 µL aliquot is then withdrawn from each sample and diluted with 900 µL of 50:50 acetonitrile:water. The pH of the filtrate is recorded using calibrated scientific pH equipment. The solution concentration of the compound is determined by HPLC using an Agilent Zorbax Bonus-RP 4.6 × 75 mm, 3.5 µm column under the following conditions: ; temperature is 30° C.; injection volume is 4 µL; ultraviolet detection at 238 nm; flow rate is 1.5 mL/min; autosampler temperature is 25° C.; mobile phase A is 0.1% trifluoroacetic acid in water; and mobile phase B is 0.1% trifluoroacetic acid in acetonitrile. The HPLC gradient is as follows: 0 min -95% A, 5% B; 9.5 min - 23% A, 77% B; 12.1 min - 23% A, 77% B; 13 min - 5% A, 95% B; 16 min - 5% A, 95% B; 16.1 min - 95% A, 5% B; 20 min - 95% A, 5% B. The table below (Table 3) details the equilibrium solubility data and equilibrium pH, reported as a mean of duplicate sample preparations. The solid form of the residual solid from the centrifuge sample is verified by XRPD, as noted in Table 5.

TABLE 5

Solubility at 37° C. following 24-hour equilibration of the crystal form of selpercatinib.

| Solvent¹ | Solubility (mg/mL) | Equilibrium pH |
|---|---|---|
| Water | 0.0036 | 7.146 |
| 0.1N HCl² | ≥ 10 | 1.297 |
| 0.01N HCl | 4.7700 | 3.634 |
| pH 4.0 Citrate/Phosphate | 0.7116 | 4.134 |
| pH 4.5 Acetate (USP) | 0.1694 | 4.496 |
| pH 6.0 Phosphate (USP) | 0.0086 | 6.027 |
| pH 7.5 Phosphate (USP) | 0.0022 | 7.526 |
| 0.01 N NaOH | 0.0011 | 9.985 |
| SGF³ | 1.440⁴ | 2.644 |
| FaSSIF⁵ | 0.0096 | 6.445 |
| FeSSIF⁶ | 0.2277 | 4.928 |

¹ Descriptions are consistent with the USP, Ph.Eur., and Japanese Pharmacopoeias.
² Solubility for 0.1 N HCI was measured at 21.5° C. (ambient).
³ SGF: Simulated gastric fluid (0.01 N HCI/Na Laurylsulfate 0.05%/NaCl 0.2%).
⁴ XRPD of retained solids exhibited non-crystalline material.
⁵ FaSSIF: Fasted-state simulated intestinal fluid (NaH₂PO₄ 28.66 mM, Na Taurocholate 3 mM, Lecithin 0.75 nM, NaCI 105.8 mM, pH 6.5).
⁶ FeSSIF: Fed-state simulated intestinal fluid (acetic acid 144.04 mM, Na Taurocholate 15 mM, Lecithin 3.75 mM, NaCl 203.17 mM, pH 5.0).

The low solubility at higher pH and moderate to high solubility in acidic media is consistent with a weak free base of low intrinsic solubility. The results indicate that the intrinsic solubility of this crystal form of selpercatinib is low (approximately 0.001 mg/mL).

What is claimed is:

1. A crystalline form of selpercatinib, which is characterized by at least one of:
(a) an x-ray powder diffraction (XRPD) pattern comprising a peak at 21.1° and peaks at 17.1°, 17.7°, and 19.8° ± 0.2° 2θ as measured using an x-ray wavelength of 1.5418 Å.

2. The crystalline form of selpercatinib according to claim 1, wherein the crystalline form is characterized by having an x-ray powder diffraction (XRPD) pattern comprising a peak at 21.1° and one or more peaks at 7.5°, 12.0°, 13.2°, 17.1°, 17.7°, and 19.8° ± 0.2° 2θ as measured using an x-ray wavelength of 1.5418 Å;.

3. The crystalline form of selpercatinib according to claim 1, wherein the crystalline form is characterized by having an x-ray powder diffraction (XRPD) pattern having characteristic peaks occurring at 7.5°, 10.9°, 12.0°, 13.2°, 17.1°, 17.7°, 18.2°, 19.8°, 21.1°, and 24.5° ± 0.2° 2θ.

4. (canceled)

5. (canceled)

6. The pharmaceutical composition comprising a crystalline form of selpercatinib according to claim 1, and a pharmaceutically acceptable carrier, diluent, or excipient.

7. The pharmaceutical composition according to claim 6, wherein the composition contains less than about 20% by wt. of other crystal forms of selpercatinib.

8. The pharmaceutical composition according to claim 6, wherein the composition contains less than about 10% by wt. of other crystal forms of selpercatinib.

9. The pharmaceutical composition according to claim 6, wherein the composition contains less than about 5% by wt. of other crystal forms of selpercatinib.

10. The method of treating cancer in a patient comprising administering to a patient in need of such treatment an effective amount of selpercatinib according to claim 1.

11. (canceled)

12. (canceled)

13.
The method according to claim 10, wherein the cancer is selected from the group consisting of: lung cancer, papillary thyroid cancer, medullary thyroid cancer, differentiated thyroid cancer, recurrent thyroid cancer, refractory differentiated thyroid cancer, multiple endocrine neoplasia type 2A or 2B (MEN2A or MEN2B, respectively), pheochromocytoma, parathyroid hyperplasia, breast cancer, colorectal cancer, papillary renal cell carcinoma, ganglioneuromatosis of the gastroenteric mucosa, and cervical cancer.

14. The method according to claim 13, wherein the cancer is medullary thyroid cancer.

15. The method according to claim 13, wherein the cancer is lung cancer and the lung cancer is small cell lung carcinoma, non-small cell lung cancer, bronchioles lung cell carcinoma, RET fusion lung cancer, or lung adenocarcinoma.

16. The method according to claim 15, wherein the cancer is RET fusion lung cancer.

17. A process for making the crystalline form of selpercatinib of claim 1, comprising the steps of:
(a) suspending selpercatinib in a solvent, wherein the solvent comprises methanol;
(b) heating the suspension to between 50° C. and 60° C. while stirring for 30 to 90 minutes;
(c) removing the heat and allowing the suspension to cool to room temperature to form solid crystals; and
(d) collecting the solid crystals.

18. (canceled)

19. (canceled)

20. (canceled)

21. (canceled)

22. A method of converting selpercatinib Form A to selpercatinib Form B, the method comprising combining selpercatinib Form A with a C₁-C₅ alcohol to generate a slurry, and isolating selpercatinib Form B from the slurry.

23. (canceled)

24. The method according to claim 22, wherein the C₁-C₅ alcohol is about 10° C. to about 30° C., and the C₁-C₅ alcohol comprises methanol.

25. (canceled)

26. (canceled)

27. (canceled)

28. (canceled)

29. The method according to claim 24, wherein C₁-C₅ alcohol comprises at least 90 wt% methanol.

30. (canceled)

31. (canceled)

32. The method according to claim 24, wherein isolating Form B comprises centrifugal separation.

33. (canceled)

34. A method of converting selpercatinib Form A to selpercatinib Form B, the method comprising:
a. dissolving the selpercatinib Form A in a solvent comprising DMSO to form a solution;
b. adding water to the solution and thereby forming a slurry;
c. isolating the selpercatinib Form B.

35. The method according to claim 34, wherein the concentration of Form A dissolved in DMSO is about 10-15 mL/g.

36. The method according to claim 34, wherein the concentration of Form A dissolved in DMSO is about 12-13 mL/g.

37. The method according to claim 34, wherein forming the solution of step a comprises heating the selpercatinib Form A and the solvent comprising DMSO to about 50° C. to about 70° C.

38. The method according to claim 37, wherein the solution is then cooled to a temperature less than about 70° C. and greater than about 20° C.

39. The method according to claim 37, wherein the solution is then cooled to a temperature of about 50° C.

40. The method according to claim 38, wherein step b comprises adding about 0.1 to about 1 mL of water per gram of Form A to the solution.

41. The method according to claim 38, wherein step b comprises adding about 0.3 mL of water per gram of Form A to the solution.

42. The method according to claim 40, wherein step b further comprises adding about 1 to about 15 wt% of Form B seed crystals.

43. (canceled)

44. (canceled)

45. The method according to claim 40, wherein the slurry is stirred for about 6 to about 72 hours, after the water is added in step b.

46. (canceled)

47. The method according to claim 40, wherein step b further comprises adding a second tranche of water to the slurry.

48. The method according to claim 47, wherein the second tranche of water comprises about 0.5 to about 3 mL of water per gram of Form A .

49. The method according to claim 34, wherein the slurry of step b is cooled to about 20-30° C.

50. (canceled)

51. The method according to claim 34, wherein the isolated selpercatinib Form B from step c is washed with a solvent comprising methanol, ACN, MTBE, or water.

52. The method according to claim 51, wherein the isolated selpercatinib Form B is washed with a solvent comprising methanol.

53. The method according to claim 52, wherein the isolated selpercatinib Form B is washed with methanol until the isolated selpercatinib Form B contains less than 0.5 wt % DMSO.

54. A method of converting selpercatinib Form A to selpercatinib Form B, the method comprising: combining selpercatinib Form A and methanol to form a slurry, and stirring the slurry until >99 wt% of the Form A is converted to Form B; wherein the concentration of the selpercatinib Form A in the methanol is about 8 mL/g.

55. (canceled)

56. (canceled)

57. A method of converting selpercatinib Form A to selpercatinib Form B, the method comprising dissolving selpercatinib Form A in DMSO at about 60-80° C. to form a solution having a concentration of about 10-15 mL/g of DMSO per gram of Form A; cooling the solution to about 40-60° C., adding the first addition of water; optionally seeding the resulting mixture with Form B seed crystals; stirring the mixture; adding the second addition of water; heating the mixture to about 60-80° C.; cooling the mixture and isolating the Form B.

58. A method according to claim 57, wherein 5 wt% of Form B seed crystals are added to the mixture.

59. The method according to claim 57, wherein the first addition of water is about 0.1 mL/g of Form A to about 0.5 mL/g of Form A.

60. The method according to claim 59, wherein the second addition of water is about 1.0-1.5 mL/g of Form A.

61. A process for preparing selpercatinib as polymorph Form B, of Formula I: 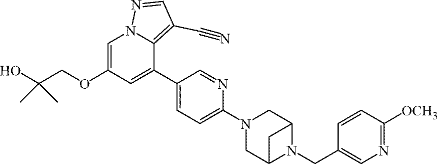 [SMILES: COc1ccc(CN2C3CC2CN(c2ccc(-c4cc(OCC(C)(C)O)cn5ncc(C#N)c45)cn2)C3)cn1]
or a pharmaceutically acceptable salt thereof,
wherein the process comprises reacting a compound of the structure: 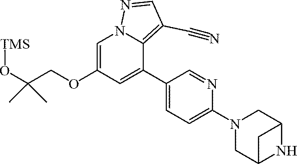 [SMILES: CC(C)(COc1cc(-c2ccc(N3CC4CC(C3)N4)nc2)c2c(C#N)cnn2c1)O[Si](C)(C)C]
or a salt thereof, in a solvent with 6-methoxynicotinaldehyde in the presence of an acid and a reducing agent to prepare selpercatinib Form B or a pharmaceutically acceptable salt thereof; wherein the solvent comprises anisole.

62. (canceled)

63. (canceled)

64. (canceled)

65. (canceled)

66. The process of claim 61, wherein the reducing agent is selected from the group consisting of sodium triacetoxyborohydride (STAB), sodium borohydride, and sodium cyanoborohydride.

67. (canceled)

68. (canceled)

69. (canceled)

70. (canceled)

71. A compound that is 4-[6-(3,6-diazabicyclo[3.1.1]heptan-3-yl)-3-pyridyl]-6-(2-methyl-2-trimethylsilyloxy-propoxy)pyrazolo[1,5-a]pyridine-3-carbonitrile, which has the structure [3] 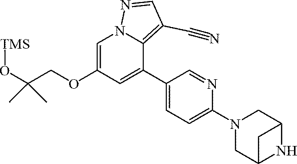 [SMILES: CC(C)(COc1cc(-c2ccc(N3CC4CC(C3)N4)nc2)c2c(C#N)cnn2c1)O[Si](C)(C)C]
or a pharmaceutically acceptable salt thereof.
